(19) United States
(12) Patent Application Publication
(10) Pub. No.: US 2009/0162477 A1
(43) Pub. Date: Jun. 25, 2009

(54) High yield maize derivatives

(76) Inventors: Daniel Nadel, Jerusalem (IL); Michael Nadel, Los Angeles, CA (US); Barry Nadel, Kfar Pinnes (IL)

Correspondence Address: ROBERT G. LEV, 4766 MICHIGAN BLVD., YOUNGSTOWN, OH, 44505, US

(21) Appl. No.: 12/004,894

(22) Filed: Dec. 21, 2007

(51) Int. Cl. A23L 1/105; A01H 1/02; A23K 1/14; C13F 1/02; C12G 3/00; A23L 1/00

(52) U.S. Cl. 426/18; 800/263; 426/416; 426/489; 127/58; 426/452

(57) ABSTRACT

A Method for producing triploid corn seeds and plants is described. The method comprises combining two parent inbreds of different ploidy levels, wherein one parent inbred is a tetraploid (4N) and the other parent is a diploid (2N) so as to produce a triploid hybrid corn seed; and cultivating the triploid hybrid corn seed to form a triploid corn plant. Usage of triploid corn plants as an economic source of sugar and ethanol is described, as is the production of molasses, rum and fodder from plant material of the low sterility triploid corn plants.

### FIELD OF THE INVENTION

The present invention relates in general to methods for producing high sugar corn plants and more particularly to genetic methods for producing high sugar corn plants and products and uses thereof.

### BACKGROUND

Corn is an important crop used as a human food source, animal fodder, silage and as a raw material in industry. The food uses of corn grain, in addition to the human consumption of corn kernels, include products of both the dry milling and wet milling industries. The principal products of dry milling include grits, meal and flour. The principal products of wet milling include starch, syrups and dextrose. “The Economic Feasibility of Ethanol Production from Sugar in the United States”, (2006) The Office of The Chief Economist, USDA.

The industrial applications of corn starch and flour are based on their functional properties, such as viscosity, film formation ability, adhesiveness, absorbent properties and ability to suspend particles. Corn starch and flour are used in the paper and textile industries and as components in adhesives, building materials, foundry binders, laundry starches, diapers, seed treatments, explosives, and oil-well muds. Starch from corn grain or seed is also used extensively in the industry as a source of sugars for producing ethanol. For example, in 2006, 4.86 billion gallons of ethanol were produced in the United States (National Corn Grower's Association, 2006). This was an increase of more than 25 percent over 2005. These numbers show that ethanol is an important fuel which is also increasingly being blended with gasoline and may be used for powering vehicles. Interestingly, Henry Ford's original horseless carriage was fuelled by corn based ethanol.

Maize plants (Zea mays L.) can be bred by both self-pollination and cross-pollination techniques. Maize has male flowers, located on the tassel, and female flowers, located on the ear, of the same plant. Natural pollination occurs in maize when wind blows pollen from the tassels to the silks that protrude from the tops of the incipient ears.

The reproductive cycle requires that pollen from a male inflorescence pollinate each individual piece of silk that is the receptor of the female inflorescence in order to develop an individual seed on the ear. With full pollination all of the silk will receive pollen and produce an entire ear of corn.

Various publications relate to methods for plant breeding/genetically manipulating corn so as to form corn with improved grain yields. For example, U.S. Pat. No. 4,705,910A, to Price, describes a hybrid tetraploid corn seed and a process for its production. Upon growth, the hybrid tetraploid corn seed yields a hybrid tetraploid corn plant, which exhibits superior biomass yield when compared to hybrid diploid corn plants. The hybrid seed is produced by crossing a first inbred line of tetraploid corn with a non-identical second inbred line of tetraploid corn to form at least one hybrid tetraploid seed.

U.S. Pat. No. 4,659,668 to Sondahl et al., describes a method for high frequency plant regeneration from somatic stem donor tissue of field grown Zea diploperennis, a diploid, perennial corn ancestor with high tillering capacity. This species is used as a parent in a maize improvement strategy to transfer the unique traits of high tillering and plantlet regeneration capacity into cultivated corn. After 3 to 4 subcultures of cultured somatic tissues on a primary medium, small callus fragments are transferred to secondary medium devoid of the auxin, 2,4-D. After a few days, numerous shoots regenerate and develop into normal plantlets which are then separated and transferred to a tertiary medium for root development. The selection of somaclonal variants form cultured somatic cells of interspecific hybrids between corn and teosinte are used for the synthesis of unique breeding lines suited for the development of improved corn varieties. A protocol for gene transfer employing recombinant DNA techniques is also described.

U.S. Pat. No. 4,810,649 to Sondahl et al., describes a method for high frequency plant regeneration from somatic stem donor tissue of field grown Zea diploperennis, a diploid, perennial corn ancestor with high tillering capacity. This species is used as a parent in a maize improvement strategy to transfer the unique traits of high tillering and plantlet regeneration capacity into cultivated corn. After 3 to 4 subcultures of cultured somatic tissues on a primary medium, small callus fragments are transferred to a secondary medium devoid of the auxin, 2,4-D. After a few days, numerous shoots regenerate and develop into normal plantlets which are then separated and transferred to a tertiary medium for root development. The selection of somaclonal variants form cultured somatic cells of interspecific hybrids between corn and teosinte are used for the synthesis of unique breeding lines suited for development of improved corn varieties. A protocol for gene transfer employing recombinant DNA techniques is also described.

US2003109011 to Hood et al., describes methods for the cost-effective saccharification of polysaccharides in lignocellulosic biomass, particularly in crop residues. In one embodiment of the invention polysaccharide-degrading enzymes are expressed in the seeds of plants, preferably in germ (embryo) tissue of seed. Corn crop plants are used in one embodiment of the invention. The corn seeds and the corn stover are harvested concurrently in a single-pass harvesting operation to lower costs. The corn seeds are fractioned which allows for additional uses of the separated tissue. For example, the endosperm can be used as a source of starch for existing industries to produce by-product credits. In one embodiment, the starch is used to produce ethanol in currently existing facilities, and the tissue, preferably germ, containing the polysaccharide-degrading enzymes can be used as the enzyme source. The appropriate tissues that express polysaccharide-degrading enzymes, or extracts thereof, are combined with the corn stover and the combination is exposed to conditions favorable for the conversion of the cell wall polysaccharides in the corn stover into fermentable sugars. The fermentable sugars can then be utilized by micro-organisms to produce ethanol or other desired fermentative products.

Triploids

As a result of breeding programs, triploids and triploid hybrids have found commercial use for various crops. Triploids of most plant species have very low fertility rates. For example, seed-free bananas may be achieved by producing triploid hybrids. Breeding banana is a difficult exercise due to complexities resulting from parthenocarpy, sterility, polyploidy and vegetative propagation. The uniqueness lies in the fact that in banana which is almost sterile, raising sexual progeny in sufficient numbers to combine desirable characters and at the same time resulting in another sterile plant is indeed very difficult. Many popular cultivated banana varieties are tetraploid, while many of the wild cultivars having desirable characteristics such as disease resistance, for example, are diploid. Triploid banana hybrids allow the breeders to incorporate important economic traits while maintaining a final product that is still sterile; it being appreciated that sterility in banana is essential to preserve its seedless characteristics. See: http://www.ikisan.com/links/ap_bananaCrop%20Improvement.shtml incorporated herein by reference.

Watermelon is another major crop where, in the recent past, breeders have turned to the commercial use of triploids. See http://edis.ifas.ufl.edu/CV006 incorporated herein by reference.

Tetraploid (4N) female inbred lines are crossed with diploid (2N) inbreds, resulting in a triploid (3N). The triploid's pollen is non-functional, and for fruit set, one needs normal diploid pollen to stimulate fruit production. The female gametes are also sterile and all that is formed inside the fruit are rudimentary white seeds with no endosperm and no embryo, with only a soft pericarp.

U.S. Pat. No. 5,007,198 to Elstrom et al., discloses a process which facilitates the rapid and economical production of seedless watermelon seed. The process described involves cloning desirable tetraploid watermelon parental lines. These parental lines are essential in the production of triploid seed for the seedless watermelon. The subject invention makes possible the use of tetraploid parental lines in the production of self-sterile triploid seed.

Research in the field of maize triploids has been centered on genetic studies, mostly concerning chromosome behaviour at different ploidy levels via cytological studies (B. McClintock: Genetics. March 1929; 14(2): 180-222). To date there have been no successful, economic use for triploid corn.

Accordingly, there remains a need for alternative methods to produce carbohydrates for food and alcohols from maize (Zea mays) plants in a more efficient and time-saving manner.

All publications cited in this application are herein incorporated by reference.

### SUMMARY OF THE INVENTION

The present invention is directed to the provision of methods for producing maize stalk having a high sugar content relative to the sugar content in stalks of maize plants of the prior art.

In another aspect, the present invention provides for producing a very low fertility female corn plant or a corn plant that has both male-sterility and very low female fertility by introgressing into a maize plant the genes for male sterility.

In another aspect, the present invention provides triploid corn plants.

The triploid corn plants of the present invention may be produced by one of the following methodologies:

A) Crossing a tetraploid (4N) plant with a diploid (2N) plant to form triploid progeny.

B) Culturing hexaploid (6N) microspores to produce resultant haploid pro-embryos (3N), andC) Regenerating haploid pro-embryos (3N) into plants.

Any of the Zea triploids (3N) related to herein may be treated with a ploidy-inducing environment or chemical, such as but not limited to heat, nitrous oxide and colchicines, to induce polyploidy and the resulting hexaploids (6N) microspores may be further cultured to produce triploid plants.

In another aspect, the present invention provides triploid corn plants which may advantageously be used as a raw material for the extraction of sugars and other carbohydrates, and for the production of ethanol and other alcohols, fermentation products produced from the fermentation of stalks, stalk extracts and stalk liquids/juice; biofarming products form triploid corn plants, useful molecules in the juice or extracts thereof, molasses, distilled spirits.

In another aspect, the present invention provides for the production of fodder having an increased level of sugar and digestibility resulting from corn plants with very low female fertility.

In another aspect, the present invention provides for reduced growing costs of corn plants due to a shortened maturity period for earless corn plants.

In another aspect, this invention provides for a sugar and/or ethanol source that provides an alternative to sweet sorghum, sugarcane and sugar beets, that may be cultivated in tropical, subtropical and temperate zones.

In another aspect, the present invention provides for multiple plantings and harvests per year using the same plot of land in subtropical, tropical and in some temperate zones as a consequence of the harvesting the corn plant prior to anthesis.

In another aspect, this invention provides for growing corn plants in much higher densities than corn plants planted for grain resulting in physiologically-induced (non-genetic) reduction in ear formation.

In another aspect, this invention provides for reduction in the energy consumption required to convert corn plant biomass into refined sugar, or molasses compared to sugarcane or sugar beet.

In another aspect, this invention provides for a reduction in both fossil fuel use and in carbon dioxide emissions generated in consequence of the production of ethanol or distilled spirits as compared to corn grain-derived ethanol production.

In another aspect, this invention provides for a reduction in water utilization per gallon of ethanol produced, as compared to corn grain-based ethanol production.

In another aspect, this invention provides for lower cost extraction of high sucrose juice relative to that obtainable from sugarcane or sugar beets.

In another aspect, this invention provides for lower cost extraction of juices in consequence of the lower amount of fiber in the interior of the maize stalk.

In another aspect, this invention provides for less fossil fuel energy and lower carbon dioxide emissions generated to extract sugar juice from the corn plants than would be required to extract a similar yield from either sugarcane or sugar beets.

In another aspect, this invention provides for selection of increased stalk or stalk mass as a trait contributing directly to increased juice yields per acre.

In another aspect, this invention provides for a lower harvest cost per kilo of sucrose per acre compared to sugarcane or sugar beet.

In another aspect, this invention provides for a higher yield of sucrose from corn plants as measured per gram of biomass, and in total biomass per acre than either sugarcane or sugar beets.

In another aspect, this invention provides for higher yield of sugars and/or ethanol per acre from corn plants compared to corn grown for grain, sugarcane or sugar beets, or sweet sorghum.

In another aspect, this invention provides for smaller plant propagation costs relative to those incurred by vegetatively-propagated sugar cane.

In another aspect, this invention enables higher planting density compared to forage corns having ears, thereby allowing higher total yields of green chop or silage.

In another aspect, this invention provides for the elimination of the need to burn plant leaves prior to harvest when compared to sugarcane.

In another aspect this invention provides for lower input growing- costs than traditional corn for grain due to early maturity.

In another aspect, this invention allows for a higher production output of ethanol per acre per day when compared to the present technology of sugarcane, sugar beets, sorghum or corn grain.

In another aspect, this invention provides for a unique method for producing maize stalk comprising a high sugar content around the time of onset of anthesis.

In another aspect, this invention provides for the production of heat energy for steam and electricity production from the dried leaves, tassels and residual bagasse from the harvesting of triploid corn plants.

In another aspect, this invention provides for reduced growing costs of corn plants due to the earlier harvesting of earless corn plants. In another aspect, this invention provides for an alternative sugar and ethanol source, relative to sweet sorghum, sugarcane and sugar beets that is suitable for cultivation in tropical, subtropical or temperate zones.

In another aspect, this invention provides for multiple plantings and harvests per year using the same plot of land in subtropical, tropical and in some temperate zones through earlier maturity of the corn plant.

In another aspect, this invention provides for growing corn plants in much higher densities than corn plants planted for grain resulting in a reduction in or a lack of, ear formation.

In another aspect, this invention provides for reduction in energy consumption required to convert corn plant biomass into refined sugar, or molasses compared to sugarcane or sugar beet.

In another aspect, this invention provides for a reduction in fossil fuel use and carbon dioxide emissions generated to produce ethanol or distilled spirits compared to corn grain ethanol production.

In another aspect, this invention provides for a reduction in water utilization per gallon of ethanol produced when compared to corn grain-based ethanol production.

In another aspect, this invention accumulates sucrose and other sugars in maize stalk which is substantially less fibrous than sugarcane, thereby permitting extraction of juice using less mechanical force and energy than required to extract juice from sugarcane.

In another aspect, this invention enables sequential mechanical harvest and on-site mechanical extraction of sugar juice from the corn plants.

In another aspect, this invention provides for lower cost extraction of high sucrose juice relative to sugarcane or sugar beets due to lack of fiber in the interior of the stalk.

In another aspect, this invention provides for less fossil fuel energy and lower carbon dioxide emissions generated to extract sugar juice from the corn plants than either sugarcane or sugar beets.

In another aspect, this invention provides for selection of increased stalk or stalk mass as a trait contributing directly to increased juice yields per acre.

In another aspect, this invention provides for a lower harvest cost per kilo of sucrose per acre compared to sugarcane or sugar beets.

In another aspect, this invention provides for a higher yield of sucrose from corn plants as measured by per gram of biomass, and in total biomass per acre than either sugarcane or sugar beets.

In another aspect, this invention provides for higher yield of sugars, and ethanol per acre from corn plants compared to corn grown for grain, sugarcane or sugar beets, or sweet sorghum.

In another aspect, this invention provides for single-stalked and tillerless corn plants at higher plant populations per acre than is possible with sugarcane.

In another aspect, this invention enables higher planting density compared to forage corns having ears, allowing higher total yields of green fodder or silage.

In another aspect, this invention provides for the elimination of the need to burn plant leaves prior to harvest when compared to sugarcane.

In another aspect, this invention allows for a higher production output of ethanol per acre day when compared to sugarcane, sugar beets, sorghum or corn grain.

Thus a preferred aspect of the present invention is directed to providing a method for producing a high carbohydrate triploid corn plant, the method comprising:
(a) combining two parent inbreds of different ploidy levels, wherein one parent inbred is a tetraploid (4N) and the other parent is a diploid (2N) so as to produce a triploid hybrid corn seed; and
(b) cultivating said triploid hybrid corn seed to form a triploid corn plant.

Optionally, the combining step (a) comprises crossing a tetraploid female with a male diploid to form the triploid hybrid corn seed.

Alternatively, the combining step (a) comprises crossing a tetraploid male with a diploid female to form the triploid hybrid corn seed.

Typically, the triploid corn plant has a female fertility level of less than 20%.

Preferably, the triploid corn plant has a female fertility level of less than 10%.

Optionally and preferably, the female fertility level enhances carbohydrate storage in stalks of said triploid corn plant.

In preferred embodiments, the triploid corn plant has at least one of the following properties:
i. less than 20% of seed count of a normal diploid corn plant; and
ii. a calorific value of around 10-15% less than an earless diploid corn plant.

Preferably, a stalk of the triploid corn plant comprises between 10% and 90% more carbohydrates than a stalk of a normal diploid corn plant.

Typically, the carbohydrate comprises at least one sugar.

Typically, the at least one sugar is selected from the group comprising monosaccharides and disaccharides.

Typically, the at least one sugar comprises sucrose.

Preferably, the triploid corn plant comprises at least 3% sucrose.

Preferably, a liquid extract of stalks from said triploid corn plant comprises at least 15° Brix.

Optionally, the sucrose yield per acre of a crop of the triploid corn plant is at least 25% more than a sucrose yield per acre of a crop of a normal diploid corn plant.

Optionally, the method further comprises the step of producing fodder from the triploid plants.

In preferred embodiments, the fodder has an increased level of digestibility, relative to fodder produced from normal diploid corn plants.

Optionally, the cultivating step comprises planting a plurality of said triploid hybrid corn seeds at an average spacing of less than 0.4 meter.

In preferred embodiments, the triploid corn plant matures within 100 days.

In some embodiments, methods of the invention further comprise extracting juice from stalks of said triploid plant.

In some embodiments, methods of the invention further comprise the additional step of fermenting the juice.

In other embodiments, methods of the invention further comprise the step of producing molasses from the juice.

In some embodiments, methods of the invention further comprise the additional step of distilling said at least one fermentation product to produce spirits.

In some embodiments, methods of the invention further comprise the additional step of crystallizing sugar from the juice.

In some embodiments, methods of the invention further comprise the additional step of producing corn bagasse from the stalks.

Another aspect of the invention is directed to a method for producing alcohols comprising the steps of:
(a) producing the triploid corn plant according to the method of claim 1;
(b) extracting juice from stalks of the triploid corn plant; and
(c) fermenting the juice to produce at least one alcohol.

Typically, the at least one alcohol comprises ethanol.

A further aspect of the invention is directed to a triploid corn plant produced in accordance with the method of:
(a) combining two parent inbreds of different ploidy levels, wherein one parent inbred is a tetraploid (4N) and the other parent is a diploid (2N) so as to produce a triploid hybrid corn seed; and
(b) cultivating said triploid hybrid corn seed to form a triploid corn plant.

A further aspect of the invention is directed to providing a cytoplasmic male sterile triploid corn plant.

A further aspect of the invention is directed to providing cytoplasmic male sterile triploid plant in accordance with the method of:
(a) combining two parent inbreds of different ploidy levels, wherein one parent inbred is a tetraploid (4N) and the other parent is a diploid (2N) so as to produce a triploid hybrid corn seed; and
(b) cultivating said triploid hybrid corn seed to form a triploid corn plant produced by combining two parent inbreds of different ploidy levels, wherein one parent inbred is a tetraploid (4N) and the other parent is a diploid (2N) so as to produce a triploid hybrid corn seed, wherein a female parent carries a cytoplasmic male sterility gene.

A further aspect of the invention is directed to a method for producing a high carbohydrate triploid corn plant, the method comprising:
(a) combining two parent inbreds of different ploidy levels, wherein one parent inbred is a tetraploid (4N) and the other parent is a diploid (2N) so as to produce a triploid hybrid corn seed, wherein a female parent carries a cytoplasmic male sterility gene; and
(b) cultivating said triploid hybrid corn seed to form a triploid corn plant.

Typically said triploid corn plant has a female fertility level of less than 10%.

Further aspects of the invention will become apparent in the description and claims that follow. In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Allele. An allele is any of one or more alternative forms of a gene, all of which relate to one trait or characteristic. In a diploid cell or organism, the two alleles of a given gene occupy corresponding loci on a pair of homologous chromosomes.

Anthesis. Anthesis means the period or act of flowering.

Backcross. Backcross is the cross of a hybrid to either one of its parents. The offspring of such a cross is referred to as the backcross generation.

Bagasse. Bagasse means the biomass remaining after corn stalks are crushed to extract their juice. Bagasse is often used as a primary fuel source for sugar mills; when burned in quantity, bagasse produces sufficient heat energy to supply all the needs of a typical sugar mill, with energy to spare.

Biopharming The application of genetic engineering on living organisms to induce or increase the production of pharmacologically active substances.

Brix (Bx). Brix (Bx) is a measurement of the mass ratio of dissolved sucrose to water in a liquid. Brix is measured with a saccharimeter that measures specific gravity of a liquid or is measured with a refractometer. A 25°Bx solution has 25 grams of sucrose sugar per 100 grams of liquid. That is, there are 25 grams of sucrose sugar and 75 grams of water in the 100 grams of solution.

Close proximity means at a greater planting density than currently practiced for corn grain production. For corn grain production, up to around 30,000 plants per acre is the typical plant density commercially grown. Consequently, the term close proximity as used herein is indicative of a planting density of higher than 35,000 plants per acre. In preferred embodiments, planting densities of 60,000 plants per acre or more are obtainable.

Corn. Corn, also known as maize includes any and all inbred, hybrid, progeny or population of Zea mays L. and its subspecies.

Stalk. The stalk or stalks of a maize plant excluding the leaves, inflorescences and roots.

Decreased Maturity Date. Decreased maturity date means precocious maturation or earlier than normal maturation.

Earless corn. Earless corn means any inbred, hybrid, progeny or population of Zea mays L. that does not produce ears consisting of husks, silk, seed and cob.

Embryo. The embryo is the rudimentary plant in a seed. The embryo arises from the zygote.

Endosperm. Endosperm means the nutritive tissue formed within the embryo sac in seed plants. It commonly arises following the fertilization of the two primary endosperm nuclei of the embryo sac by the two male sperm. In a diploid organism the endosperm is triploid.

Ethanol. Ethanol is a flammable, colorless chemical compound, also known as ethyl alcohol and grain alcohol. As a biofuel, ethanol is a clean-burning, high-octane fuel that may be produced from renewable sources such as corn plants.

Ethanol per acre day. The unit Ethanol per acre day is a means of quantifying the total potential ethanol output of a crop per acre divided by the average number of days to harvest. This provides a means to compare different crops for their efficiency as sources of ethanol.

Extraction. Extraction as used herein relates to a method of for separating sucrose bearing juice from corn stalks. Typically, roller mills are used for this purpose.

Female Reproductive Structure. Female reproductive structure means the female gametes and those portions of the plant that are specialized for the production, maturation and viability of female gametes. Female reproductive structures, which can be an ear of maize, or those portions of a plant that produce the carpel and/or the gynoecium (pistil). The carpel of a plant includes but is not limited to, a stigma, style, ovary and cells or tissues thereof.

Female-Sterile Plant. As used herein, the term female-sterile plant relates to a plant that is incapable of producing viable seed when pollinated with functional or viable pollen. Such female sterility may be the result of breeding selection and/or the presence of a transgene. A “conditionally female-sterile plant” refers to a plant which under normal growing conditions is female fertile and which can become female-sterile under specific conditions. In the context of the current invention, examples of these conditions include the exogenous application of a pro-herbicide or other non-phytotoxic substance, for example. In the context of the current invention such a “female-sterile plant” or “conditionally female-sterile plant” may remain male-fertile and able to produce viable pollen.

Female-Sterile and Male-Sterile Corn Plant. The term female-sterile and male-sterile plant is used herein to describe a corn plant that is both female-sterile and male-sterile and is thus both incapable of producing viable seed or viable pollen.

Fermentable sugars. The term fermentable sugars relates to hexose sugars including but not limited to dextrose, glucose, galactose and fructose.

Fermentation. Fermentation is the anaerobic metabolic breakdown of a nutrient molecule, such as glucose, without net oxidation. Fermentation does not release all the available energy in a molecule; it merely allows glycolysis (a process that yields two ATP per glucose) to continue by replenishing reduced coenzymes. Depending on which organism metabolizes the nutrient molecule, fermentation may yield lactate, acetic acid, ethanol, or other reduced metabolites.

Field Corn. The term Field corn relates to corn hybrids, varieties or cultivars of corn grown extensively on large acreages within a broad but defined geographic area for the production of grain and/or forage. Field corn in the United States is also referred to as “dent” corn, whereas field corn produced in Europe and Argentina is more likely to be referred to as “flint” or “flint-dent” corn.

First Juice Press. The term First juice press as used herein, is the juice derived from the corn stalks from the first pass through the roller mills.

Fodder or animal feed, is any foodstuff that is used specifically to feed livestock. More specifically, the term Fodder as used herein generally means the above-ground portion of a corn plant including stalks, leaves and tassels harvested per unit area.

Gene. A gene refers to any DNA sequence comprising several operably linked DNA fragments such as a promoter and a 5′ regulatory region, a coding sequence and an untranslated 3′ region comprising a polyadenylation site.

Grain. The term grain as used herein relates to mature corn kernels produced by commercial growers either for on-farm use or for sale to customers; in both cases for purposes other than growing or reproducing the species. Typical customers of corn grain include livestock feeders, wet millers, dry millers and animal feed formulators.

Gross tons per acre. As used herein, the term gross tons per acre means the total weight of all corn plants stalks cut at ground level, including the tassels and leaves, per acre.

Harvesting. Harvesting is the act or process of gathering a crop.

Heterozygous. Heterozygous means a genetic condition existing when different alleles reside at corresponding loci on homologous chromosomes.

Homozygous. Homozygous means a genetic condition existing when identical alleles reside at corresponding loci on homologous chromosomes.

Hybrid. Hybrid means any offspring of a cross between two genetically unlike individuals (Rieger R., A. Michaelis and M. M. Green, 1968, A Glossary of Genetics and Cytogenetics, Springer-Verlag, N.Y.).

Hybrid Corn: Any genotype having two or more parents including the following examples:

(A) a single cross, i.e., a first generation of cross between two inbred lines;

(B) a double cross, i.e., the first generation of a cross between two single crosses;

(C) a three way cross, i.e., the first generation of a cross between a single cross and an inbred line; or

(D) a top cross, i.e., the first generation of a cross between an inbred line and an open pollinated variety, or the first generation of a cross between a single cross and an open pollinated variety.

Inbred. The term inbred refers to a substantially homozygous individual plant or variety.

Inter-planting. Inter-planting refers to a method of planting seeds or plants in a field that ensures adequate cross-pollination of male-sterile or conditionally male-sterile plants by the male-fertile plants. This can be achieved either by random mixing of female and male parent seed in different blends (80/20; 90/10; etc) before planting or by planting in specific field patterns whereby different seeds are alternated. When separate harvesting from different plants is required, planting in alternating blocks or rows is preferred.

Juice. The term juice as used herein, means the liquid bearing sugar found within the corn stalk.

Kernel. The term kernel as used herein means the corn caryopsis comprising a mature embryo and endosperm which are products of double fertilization.

Maize. Maize, also known as corn, means any inbred, hybrid, progeny or population of Zea mays L. and all of its subspecies.

Male Reproductive Structure. The male reproductive structure of corn implies the male gametes and those portions of the plant that are specialized for the production, maturation and viability of male gametes. This comprises those portions of a plant that comprise, for example, microspores, stamens, tapetum, anthers and the pollen.

Male-Sterile Plant. Male-sterile plants are plants that are incapable of supporting viable pollen formation. Such male sterility can be the result of breeding selection or the presence of a transgene. A “conditionally male-sterile plant” refers to a plant which under normal growing conditions is male-fertile and which can become male-sterile under specific conditions. Such conditions might comprise physical emasculation or application of a specific chemical gametocide. In the context of the current invention the said conditions particularly comprise the exogenous application of a pro-herbicide or other non-phytotoxic substance. In the context of the current invention such a “male-sterile plant” or “conditionally male-sterile plant” remains female-fertile and able to produce viable seeds when pollinated with functional or viable pollen.

Milk Line. The dividing line on the kernel between the harder the top portion and the milky lower portion is known as the milk line.

Non-Phytotoxic Substances. The term non-phytotoxic substances as used herein relates to substances which are relatively non-phytotoxic to plants, cells or tissues of any particular crop to which a method according to the invention is applied. Thus a non-phytotoxic substance need not be non-phytotoxic in all plant tissues of all plants. Non-phytotoxic substances include pro-herbicides. Pro-herbicides are substances with no appreciable direct toxic effect on plant tissues but which are progenitors of active phyto-toxins. In susceptible plant species, such pro-herbicides act indirectly as herbicides through the action of endogenous enzymes which convert them in planta to a phyto-toxin.

Net energy ratio relates to a concept that is important in energy economics, and refers to a surplus condition in the ratio between the energy provided by an energy source divided by the energy required to harvest the energy source.

Net weight. The term Net weight as used herein means the weight in kilograms of the corn plants stalks, excluding the tassels and the leaves.

Percentage dry matter. The term Percentage dry matter, as used herein, means the total weight of dry matter divided by the total weight of corn stalks, excluding leaves and tassels.

Percentage of sucrose. The term Percentage of sucrose as used herein, is the percentage of sucrose present in the juice extracted from corn stalks without the leaves and tassels.

Percentage purity of juice. The term percentage purity of juice as used herein, is the percent of juice extracted from the corn stalks that is not particulate matter from the stalk, dust or other foreign material.

Photoperiod. Photoperiod refers to the relationship between the length of light and dark in a 24 hour period. At the equator, day length is 12 hours of light and 12 hours of dark. In other latitudes, the photoperiod varies with the seasons.

Photoperiodic maize. Photoperiodic maize refers to the inflorescence deficiency of maize originating in tropical lowland (below 1200 meters) at short-day latitudes that is induced by the longer day length period when the tropical lowland maize is grown in spring and summer of temperate latitudes.

Photoperiod-sensitive plants are plants that display traits or characteristics whose phenotypic expression is altered by daylength.

Plant Density. The term Plant density is a measurement of the number of corn plants per unit area after planting. As used herein, the plant density is measured in thousands of plants per acre.

Plant Material. Plant material consists of all vegetative matter above the soil.

Residual plant material. The term Residual plant material as used herein implies all of the plant material remaining after extraction of the juice.

Second Juice Press. The term Second juice press as used herein, is the juice derived from the corn stalks from the second pass through the roller mills.

Seed. The term seed as used herein is the mature corn kernel produced for the purpose of propagating the species. Alternately, it is a corn kernel commonly sold to commercial grain producers or growers.

Sorghum. Sorghum bicolor (L) Moench and related families of sorghum subspecies is of a genus of numerous species of grasses, some of which are raised for grain and many of which are utilized as fodder plants either cultivated or as part of pasture. Sweet sorghum is also used to produce molasses, ethanol and distilled spirits.

Stalk. The term stalk, as used herein, refers to the stem of a corn plant. including any residual leaves and tassels attached to the stalk.

Stalk Dry Matter. The term Stalk dry matter as used herein, relates to the net dry bagasse produced after extracting the juice from the corn stalks.

Stalk Yield. The term Stalk yield as used herein, is the yield of corn stalk in tons per acre.

Sucrose Yield. Sucrose yield is the amount of sucrose expressed in tons per acre as a percentage of tons of corn stalks harvested, excluding leaves and tassels.

Sugar. Sugar means any of a class of water-soluble crystalline carbohydrates, including but not limited to, sucrose and lactose, having a characteristically sweet taste and classified as monosaccharides, disaccharides, and trisaccharides and also includes any sugar alcohols derived from monosaccharides and disaccharides.

Sugar content. The concentration of sugar per unit area. In corn for example, the sugar content can be measured using Brix readings.

Tassel. An inflorescence of male flowers producing stamens usually located at the apex of the corn plant.

Tasselless corn. Tasselless corn means any inbred, hybrid, progeny or population of Zea mays L. that does not produce a male inflorescence.

Total gross weight of 5 stalks. Total gross weight of 5 stalks means the weight of the corn plant's stalks when cut at ground level and including the tassels and leaves.

Tons of dry matter per acre. Tons of dry matter per acre means the total weight of dry matter of stalks plus the dry matter of leaves and tassels per combined acre in tons.

Tons of stalk per acre. Tons of stalk per acre means the number of tons per acre of corn plant stalks, excluding the leaves and tassels.

Volume Juice % (percentage). Volume juice percentage means the weight of juice extracted, in tons, divided by the number of tons of corn stalks harvested, excluding leaves and tassels.

Wet Leaf Mass. The wet leaf mass means the total quantity of leaves and tassels as measured in tons per acre that is available as fodder.

Wet Mass of Sample. The wet mass of sample means the weight of five stalks of earless corn, including the leaves, tassels, stalk and juice.

### DETAILED DESCRIPTION OF THE INVENTION

The following embodiments and aspects thereof are described in conjunction with systems, tools and methods which are meant to be exemplary, illustrative and not limiting in scope. In various embodiments, one or more of the above-mentioned described problems have been reduced or eliminated, while other embodiments are directed to other improvements.

Embodiments of the present invention are directed to methods for producing triploid corn/maize plants, having very low female fertility, which in turn increases sugar accumulation in the plant's stalk relative normal diploid eared corn.

In typical corn plants that produce ears, studies have shown that the sugar content of the maize stem is reported to decline during the period of rapid kernel growth. Hume, D. J., et al., “Accumulation and translocation of soluble solids in corn stalks” Can. J. Plant Sci. 52:363-368 (1972). Other studies have confirmed that corn kernels serve as the sugar reserves or “sugar sinks” of the corn plant. Felkner, F. C., et al., “Sugar Uptake by Maize Endosperm Suspension Cultures” Plant Physiol. 88:1235-1239 (1988); Chourey, P. S., et al., “Genetic control of cell wall invertases in developing endosperms of maize” Planta 223: 159-167 (2006); Ronginedefekete, M. A., et al. “Mechanism of Glucose Transfer from Sucrose into the Starch Granule of Sweet Corn” Arch Biochem Biophys. 104:173-184 (January 1964); Dickinson, David B. et al., “Presence of ADP-Glucose Pyrophosphorylase in Shrunken-2 and Brittle-2 Mutants of Maize Endosperm” Plant Physiol. 44(7):1058-1062 (July 1969).

It has been shown via manual removal of ears, that one of the effects is accumulation of sugars in the stalk as reported and confirmed by Christensen, Leslie E, et, al., “The Effects of Ear Removal on Senescence and Metabolism of Maize” Plant Physiol. 68(5):1180-1185 (November 1981); Crafts-Brandner, Steven J., et. al. “Differential Senescence of Maize Hybrids following Ear Removal” Plant Physiol. 74(2):368-373 (February 1984); Singleton, Ralph W, “Segregation for sucrose production in corn stalks”. MNL 23:8-11 (1949); and Shibuya, Tsunetoshi, “Studies of sugar production in corn stalk” MNL 25/29; Singleton, W. R., “Sucrose in the stalks of maize inbreds” Sci. 107:174. (1948).

The present invention encompasses corn plant material having a sugar content of about 6%, 6.3%, 7%, 7.5%, 8%, 8.1%, 9%, 9.6%, 10%, 10.5%, 11%, 11.2%, 12%, 12.7%, 13%, 13.6%, 14%, 14.4%, 15%, 15.9%, 16%, 16.7% and 17.0% or higher. The present invention also encompasses corn plant material having a sugar content ranging for example, between 6% to 9%, between 6.5% to 14.4% or between 7.0% to 17.0%.

The stalks of the triploid corn plants of the present invention, typically have a sugar content of 14-21°Brix, which is 20-90% greater than that normal diploid eared corn.

The triploid plants of the present invention may be planted in close proximity. This results in a plant density in the range of from 40,000 up to 80,000 plants/acre, whereas commercial corn grown for grain is typically planted at densities of from 20-35,000 plants/acre

The present invention also encompasses a corn plant density, the number of plants per acre ranging between any of the following numbers, of 41,000 plants, 42,000 plants, 43,000 plants, 44,000 plants, 45,000 plants, 46,000 plants, 47,000 plants, 48,000 plants, 49,000 plants, 50,000 plants, 51,000 plants, 52,000 plants, 53,000 plants, 54,000 plants, 55,000 plants, 56,000, plants, 57,000 plants, 58,000 plants, 59,000 plants, 60,000 plants, 61,000 plants, 62,000 plants, 63,000 plants, 64,000 plants, 65,000 plants, 66,000 plants, 67,000 plants, 68,000 plants, 69,000 plants, 70,000 plants, 71,000 plants, 72,000 plants, 73,000 plants, 74,000 plants, 75,000 plants, 76,000 plants, 77,000 plants, 78,000 plants, 79,000 plants, 80,000 plants and 81,000 plants. The present invention also encompasses a corn plant density ranging for example, between 41,000 plants and 55,000 plants, between 42,000 plants and 68,000 plants or between 45,000 plants and 81,000 plants.

This close proximity coupled with the increased sugar yields per plant provides a potential yield of ethanol or other energy fuels of 5 times that attained by conventional diploid eared corn crops.

Thus, aspects of the present invention provide a sustainable energy source and have the potential of making a significant contribution to providing Mankind's energy requirements. The potential is quantified in Tables 1 to 12 hereinbelow.

The present invention encompasses corn plant material harvested per acre per crop sufficient to produce gallons of ethanol per acre of about 470-2400 gallons/acre. In some cases the yield of ethanol per acre may be around 630 gallons, 710 gallons, 750 gallons, 787 gallons, 800 gallons, 807 gallons, 850 gallons, 863 gallons, 900 gallons, 922 gallons, 950 gallons, 1000 gallons, 1026 gallons, 1050 gallons, 1074 gallons, 1100 gallons, 1133 gallons, 1150 gallons, 1153 gallons, 1200 gallons, 1202 gallons and 1250 gallons of ethanol per acre or more.

It was unexpectedly found that in trials of hybrid earless corn plants, Brix readings ranging between 10.7% and 20% and sucrose levels ranging between 6.9% and 17.8% in the stalk were obtained.

An aspect of the present invention encompasses juice derived from corn plant material having a Brix value in the range of 10.7% to 20.0% or higher. Typical juices have brix values in the range of, for example, between 10.7% to 16.8%, between 10.9% to 19.2% or between 10.7% to 20.0%.

A further aspect of the invention encompasses sucrose levels in juice of from about 6.9% to 18% or more. Particular embodiments encompass sucrose levels in juice ranging for example, between 6.9% and 17.8%, between 7.1% and 16.7% or between 6.9% and 23.0%.

An important aspect of some embodiments of the present invention is that the triploid corn may be integrated into one or more of the following corn types: short day corn, intermediated day corn and long day corn. This feature allows for triploid seed production at the corn-growing location, eliminating the seed transportation costs that would be required to plant short-day (low latitude) corn seed in long-day (high latitude) locations.

The triploid corn plants of the present invention may be harvested after about 3 months, and typically between 90 and 100 days after planting, whereas, the short-day corn planted in a long-day area is harvested in a longer period, typically in the range of from about 110 to 180 days from planting, depending on specific strain and weather conditions. (See F. Below, 2007).

It will be appreciated that the stalks of the triploid corn plants of the present invention can be used as a feedstock for the production of alcohols such as ethanol, which may be used as a biofuel, as an industrial chemical or even as a beverage. It will be noted that this biofuel provides an alternative to the commonly used commodity fossil fuels, and has various advantages, in that it can be locally produced in many areas.

Another advantage of embodiments of the present invention is that corn plant material thereof yields a greater amount of biofuel per acre than conventional corn grain-derived ethanol. This is discussed at length, hereinbelow.

Typically, the present invention provides a greater economic return/acre than conventional corn grain production. Since in embodiments of the invention, the corn plant is not being harvested for the ears and grain, the crop matures substantially earlier (90-100 days) than a crop that is harvested for the ears and grain (130-160 days). In the Midwest, Western and Southern states of the United States, this allows for the production of a double crop each year. In warmer areas such as Brazil, Colombia, Hawaii, Florida or India, this allows for continuous year-round production, potentially producing 2 to 4 crops per year. This enables a potential yield of alcohol/acre/year of 2000-6000 gallons. Additionally, the shorter life cycle enables alternation of corn and other crops, such as legumes, for example, in the same year, and/or alternation or rotation of seed corn and seedless corn of the present invention, within a shortened time frame. In some cases of carefully selected improved triploids, the yield may reach or exceed 7000 gallons.

Embodiments of the present invention may be adapted to all current world corn production areas.

Another advantage of the present invention is that the increased level of sugar in the stalks of corn plants results in increased digestibility of the silage, or green fodder produced therefrom. Additionally, corn grain and cobs, which are less digestible than sugar are substantially eliminated.

Another advantage of the present invention when compared to sugarcane, is that it does not require leaf-burning prior to harvesting. This has a three-fold benefit: (i) the entire corn plant material is used for energy production or silage, (ii) CO₂ emissions are reduced and (iii) assuming that juice is extracted in the field and not sold as cane to local processing plants, the growers can have a dual income from the production of sugar/ethanol and silage from the leaves, tassels and bagasse or from the sale of the leaves, tassels, and bagasse for use as fuel.

Another advantage of the present invention is that in comparison to sugarcane, less energy and mechanical pressure is required to extract the juice from the plant material due to corn stalks having a soft, pithy internal structure.

### FURTHER EMBODIMENTS OF THE INVENTION

### Corn Kernels/Seed as Sugar Sinks

Corn grain or seed is the final product in the reproductive cycle of corn. After the initial vegetative growth achieves sexual maturity, male and female inflorescences are produced by the plant. This process requires the transport of sugars from storage in the stalk to be used to create the male and female inflorescences and the production of viable pollen and embryonic seeds. The reproductive process utilizes a very significant percent of the stored sugar to create seed. C. Y. Tsai, et. al., “Enzymes of Carbohydrate Metabolism in the Developing Endosperm of Maize”; Creech, R. G., “Genetic Control of Carbohydrate Synthesis in Maize Endosperm” Genetics 52:1175-1186 (1965); Makela, P., et. al., “Imaging and Quantifying Carbohydrate Transfer to the Developing Ovaries of Maize” Annals of Botany 96(5):939-949 (2005);

The production of an ear of corn requires many different energy sinks which filter the efficiency of storing carbohydrate energy in the form of seed. Those energy sinks include creation of the male inflorescence (tassel), creation of viable pollen, creation of the female inflorescence (ear), which includes, cob, silk, husk, embryos, fertilized embryos, husks, and shank. Each of these structures requires the use of sugars derived from photosynthesis and stored in the stalk for future use in vegetative and reproductive growth. McLaughlin J., et. al., “Glucose Localization in Maize Ovaries When Kernel Number Decreases at Low Potential and Sucrose is Fed to the Stems” Annals of Botany 94(1):75-86 (July 2004); Salvador, R. J., et. al., “Proposed Standar System of Nomenclature for Maize Grain Filling Events and Concepts” Maydica 40(2)141-146.

Each structure created to facilitate the reproductive cycle represents an energy sink that lowers the total carbohydrate pool available to the plant to convert into seed.

### Corn Grain as a Source of Sugar, Ethanol, Silage and Other Derived Products

Corn for silage is typically harvested at 60-70% moisture. This is usually determined when the milk line is 50-66% over the way down the immature kernels depending upon the latitude, local microclimates, and varieties of corn used maturity ranges from 95-115 days.

Corn grain requires several steps to be converted into ethanol. It is first converted to starch by wet or dry milling processes. It is then converted from starch to high fructose corn syrup, and the high fructose corn syrup is diluted with water and then fermented. After fermentation, the liquids are distilled to produce ethanol spirits. The energy for each processing step normally is provided by fossil fuels.

Processing corn grain into ethanol consumes 3-5 gallons of water for each gallon of ethanol produced. Keeney, David, et. al., “Water Use in Ethanol Plants: Potential Challenges, The Institute for Agriculture and Trade Policy” (2006); Moreira, Jose Roberto, “Water Use and Impacts Due Ethanol Production in Brazil” 1 National Reference Center on Biomass, Institute of Electrotechnology and Energy—CENBIO/IEE, University of São Paulo, São Paulo, Brazil. This generates a need for waste water treatment processes, which are also typically powered by fossil fuels.

Corn grain is the largest single crop in the USA with plantings of approximately 90 million acres in 2007. It is widely adapted for day length and can be grown commercially in all states excepting Alaska. It can be successfully cultivated between 0° to 43° latitude, which encompasses a very wide range of prime agricultural land.

Though corn grain is a fungible product, it may be stored for extended periods of time. It is easily transportable in bulk from production areas to storage silos or processing plants. There is very extensive infrastructure in place worldwide for storing and transporting corn grain to the marketplace.

Corn grain and its derivatives are used in thousands of different products and processes. Industrial uses of seed corn include use of corn starch produced by wet milling of corn grain or seed, corn flour produced by dry milling of corn grain or seed and whole kernel fermentation which is used in the production of both food-grade and industrial ethanol.

Production of corn for grain averaged 147.9 bushels per acre in the USA in 2005. Yields rise with improved genetics but are susceptible to inclement weather during the growing season.

A bushel of corn weighs 56 pounds and may supply approximately 2.75-2.90 gallons of ethanol, depending on the processing method used. On average, approximately 407-429 gallons of ethanol can be produced from an acre of corn grain produced in the USA. Corn grain takes approximately 160 days to mature.

Corn grain is covered by a tough pericarp which protects the seed. The pericarp is indigestible by humans and cattle. Corn which is not adequately dehulled or cracked will pass through a cow's stomach undigested, which limits the caloric value of whole grained corn whether dry or in the form of silage. Silage is not ground fine enough to open the majority of immature seed on the cobs at time of harvest. Hence, a significant percent of the grain passes through the animal and remains intact.

### Sugar Beets as a Source of Sugar, Ethanol, Silage and Other Derived Products

Sugar beets (Beta vulgaris L.) are cultivated in the USA as a source of sucrose and molasses and can also be used for the production of ethanol.

Sugar beets can be cultivated from approximately 30-55 degrees latitude.

Sugar beets can be stored for months if necessary, however, due to their cumbersome shape and size they are generally used for sugar production immediately after harvest.

Sucrose is extracted from sugar beets using the diffusion method which, after washing and shredding the sugar beets, adds water to them as a carrying agent. The mixture is centrifuged to extract the sugars present.

US sugar beet production averaged 22.2 tons per acre in 2005 with a recovery of 15.8% sucrose or 3.5 tons per acre. Based upon the US Department of Energy's theoretical Ethanol Yield Calculator, this would produce approximately 606 gallons of ethanol per acre. Sugar beets mature in approximately 220 days allowing the production of approximately 2.75 gallons of ethanol per acre day.

### Sugarcane as a Source of Sugar, Ethanol, Silage and Other Derived Products

Sugarcane (Saccharum species) is cultivated in the USA as a source of sucrose and molasses and can also be used for the production of ethanol.

Sugarcane is cultivated between 0 and 32 degrees latitude with the majority of production located in the tropics below 25 degrees latitude. Subsequently production of sugarcane in the USA is limited to Florida, Louisiana, Texas and Hawaii.

Sugarcane production in the USA in 2005 encompassed 922,000 acres. It averaged 28.8 tons per acre with a recovery of approximately 12.33% sucrose for 3.55 tons per acre. Based upon the US Department of Energy's Theoretical Ethanol Yield Calculator, this would produce approximately 613 gallons of ethanol per acre. Sugarcane matures in approximately 360 days allowing it to produce approximately 1.70 gallons of ethanol per acre day.

Sugarcane requires processing immediately after harvest and possesses sucrose in the juice inside the stalk. The juice is extracted by passing the sugarcane stalks through a three roller mill or first through a shredder and then through a mill. The residual dry stalk material (bagasse) is widely used as a fuel for producing all of the steam and electricity needed to process either finished sugar or ethanol. Neither corn grain nor sugar beets provide any portion of the energy used in their processing. Sugarcane provides a positive net energy and CO₂ balance, which offsets the lower number of gallons per acre day than either sugar beets or corn.

Sugar beets and sugarcane are principally grown in the USA to produce sucrose. Corn grain is also used to produce sweeteners in the form of high fructose corn syrup and dextrose. All three of these sources are widely used by industry and consumers as natural sweeteners with carbohydrate content.

### Sorghum as a Source of Sugar, Ethanol, Silage and Other Derived Products

Sorghum (Sorghum bicolor (L.) Moench) is a widely adapted grain, fodder, and sweetener crop that can be grown between 0° to 40° latitude. It requires considerably less water in cultivation than corn and can be grown under much more arid conditions.

Sweet sorghum produces both grain and a readily distillable sweet juice in the stalk. It matures in 115-130 days. It is capable of producing sufficient biomass and sugar to produce 400-500 gallons of ethanol per acre. This enables sweet sorghum to produce 3.85-4.35 gallons of ethanol per acre day in the USA.

Sweet sorghum is widely used as a forage crop to produce green fodder or silage. It also produces grain, which can be used for animal feed, human consumption, or for ethanol. Bian Yun Long, et al., “QTLs for sugar content of stalk in sweet sorghum (Sorghum bicolor L. Moench)”. Agricultural Sciences in China 5(10):736-744 (2006); Németh, T., “Relationships between nitrogen supply, dry matter accumulation and micro element content of silage sorghum (Sorghum bicolor L./Moench.)” Cereal Research Communications 34(1(II)):593-596 (2006); Reddy, B. V. S., et al., Special issue: “Characterization of ICRISAT-bred Sorghum hybrid parents (set I)”. International Sorghum and Millets Newsletter 47:138 (2006); Liu, Rong Hou, et al., “Ethanol fermentation of sweet sorghum stalk juice by immobilized yeast” Transactions of the Chinese Society of Agricultural Engineering 21(9):137-140 (2005); Reddy, B. V. S., et al., “Sweet sorghum—a potential alternate raw material for bio-ethanol and bio-energy” International Sorghum and Millets Newsletter 46:79-86 (2005); and Carmi, A., et al., “Effects of irrigation and plant density on yield, composition and in vitro digestibility of a new forage sorghum variety, Tal, at two maturity stages” Animal Feed Science and Technology 131(1/2):121-133 (2006).

A reliable method of controlling fertility in plants provides an opportunity for improved plant breeding techniques. This is especially true for the development of maize hybrids, which typically relies upon some type of male sterility system and sometimes a female sterility system.

### Breeding Techniques

Virtually all of the commercial corn produced in the United States is produced from hybrid seed. The production of hybrid seed first requires the development of elite corn inbred lines that possess good combining ability to produce agronomically superior hybrids. The majority of hybrid seed produced in the United States is of the single cross type, wherein two inbred lines are inter-mated, or crossed, to produce what is termed an F₁ single cross hybrid. The resulting kernels from this inter-mating are then sold as seed to commercial growers who plant the seed and harvest the second generation, or F₂ grain, for use on a farm or for commercial sale.

The development of maize hybrids requires the development of homozygous inbred lines, the crossing of these lines, and the evaluation of the crosses. Pedigree breeding and recurrent selection are two of the breeding methods used to develop inbred lines from populations. Breeding programs combine desirable traits from two or more inbred lines or various broad-based sources into breeding pools from which new inbred lines are developed by selfing and selection of desired phenotypes. A hybrid maize variety is the cross of two such inbred lines, each of which may have one or more desirable characteristics lacked by the other or which complement the other. The new inbreds are crossed with other inbred lines and the hybrids from these crosses are evaluated to determine which lines have commercial potential. The hybrid progeny of the first generation is designated F₁. In the development of hybrids only the F₁ hybrid plants are sought. The F₁ hybrid is more vigorous than its inbred parents. This hybrid vigor, or heterosis, can be manifested in many ways, including increased vegetative growth and increased yield.

Another traditional breeding technique is backcrossing. Backcross breeding has been used to transfer genes for a simply inherited, highly heritable trait into a desirable homozygous cultivar or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Backcrossing methods can be used with the present invention to improve or introduce a characteristic into the inbred line. The term “backcrossing” as used herein refers to the repeated crossing of a hybrid progeny back to the recurrent parent, i.e., backcrossing 1, 2, 3, 4, 5, 6, 7, 8 or more times to the recurrent parent. The parental corn plant that contributes the gene for the desired characteristic is termed the nonrecurrent or donor parent. This terminology refers to the fact that the nonrecurrent parent is used one time in the backcross protocol and therefore does not recur. The parental corn plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original line of interest (recurrent parent) is crossed to a second line (nonrecurrent parent) that carries the single gene of interest to be transferred. The resulting progeny from this cross are then crossed again to the recurrent parent and the process is repeated until a corn plant is obtained wherein essentially all of the desired morphological and physiological characteristics of the recurrent parent are recovered in the converted plant, in addition to the single transferred gene from the nonrecurrent parent.

Hybrid maize seed can be produced by a male sterility system incorporating manual detasseling. To produce hybrid seed, the male tassel is removed from the growing female inbred parent, which has been planted in alternating rows with the male inbred parent. Consequently, providing that there is sufficient isolation from sources of foreign maize pollen, the ears of the female inbred will be fertilized only with pollen from the male inbred. The resulting seed is therefore a hybrid and will form hybrid plants.

The natural variation in plant development can result in plants tasseling after manual detasseling is completed. Additionally, a detasseler will not completely remove the tassel of the plant. In any event, the female plant will successfully shed pollen and some female plants will be self-pollinated. This will result in seed of the female inbred being harvested along with the hybrid seed which is normally produced.

Alternatively, the female inbred can be mechanically detasseled by machine. Mechanical detasseling is approximately as reliable as hand detasseling, but is faster and less costly. However, most detasseling machines produce more damage to the plants than hand detasseling. Thus, no form of detasseling is presently entirely satisfactory, and a need continues to exist for alternatives which further reduce production costs and eliminate self-pollination in the production of hybrid seed.

A reliable system of genetic male sterility would provide advantages over detasseling. The laborious detasseling process can be avoided by using cytoplasmic male-sterile (CMS) inbreds. Plants of a CMS inbred are male sterile as a result of factors resulting from the cytoplasmic, as opposed to the nuclear, genome. Thus, this characteristic is inherited exclusively through the female parent in maize plants, since only the female provides cytoplasm to the fertilized seed. CMS plants are fertilized with pollen from another inbred that is not male-sterile. Pollen from the second inbred may or may not contribute genes that make the hybrid plants male-fertile. Usually seed from detasseled normal maize and CMS produced seed of the same hybrid must be blended to insure that adequate pollen loads are available for fertilization when the hybrid plants are grown and to insure diversity.

There may be other drawbacks to CMS. For example, there is an historically observed correlation between a specific variant of CMS and susceptibility to various crop diseases. This problem has discouraged widespread use of that CMS variant in producing hybrid maize.

Furthermore, it will be appreciated that control of female fertility has advantages. Currently, once the female inbred is rendered male sterile and the cross pollination has occurred, the male inbred plant is physically removed since any inbred seed on the plant cannot be sold and should not be released. The removal process adds an additional expense to the hybrid production costs. However, if the male inbred could be rendered female infertile, it would not be necessary to remove the rows of males, and any chance of inbred seed becoming available is reduced. Approximately 20 percent of acreage in producing a hybrid must be devoted to growing the male inbred. Therefore, hybrid seed is being produced on only 80% of the land being utilized for hybrid production.

One type of genetic male sterility is disclosed in U.S. Pat. Nos. 4,654,465 and 4,727,219 to Brar, et al. However, this form of genetic male sterility requires maintenance of multiple mutant genes at separate locations within the genome and requires a complex marker system to track the genes and make use of the system convenient. Patterson also described a genic system of chromosomal translocations which can be effective, but which are complicated. See U.S. Pat. Nos. 3,861,709 and 3,710,511.

Many other attempts have been made to improve on these drawbacks. For example, Fabijanski, et al., developed several methods of causing male sterility in plants (see EPO 89/3010153.8 publication No. 329,308 and PCT application PCT/CA90/00037 published as WO 90/08828). One method includes delivering into the plant a gene encoding a cytotoxic substance associated with a male tissue specific promoter. Another involves an antisense system in which a gene critical to fertility is identified and an antisense to the gene inserted in the plant. Mariani, et al. also shows several cytotoxin encoding gene sequences, along with male tissue specific promoters and mentions an antisense system. See EP 89/401,194. Still other systems use “repressor” genes which inhibit the expression of another gene critical to male sterility. PCT/GB90/00102, published as WO 90/08829.

Another system useful in controlling male sterility makes use of gametocides. Gametocides are not a genetic system, but rather a topical application of chemicals. These chemicals affect cells that are critical to male fertility. The application of these chemicals affects fertility in the plants only for the growing season in which the gametocide is applied (see Carlson, Glenn R., U.S. Pat. No. 4,936,904, which is incorporated herein by reference). Application of the gametocide, timing of the application and genotype specificity often limit the usefulness of the approach.

There has been little previous incentive to control female fertility, and thus little work in this area. One example is the work by De Greef et al described in European patent publication 0412006; U.S. Pat. No. 5,633,441. There, it was noted that female sterility was useful for producing fruit without seeds, enhanced vegetative biomass production and more flower setting within one season. The system set forth there is much like the male sterility system previously employed: the plant is transformed with a female tissue specific promoter linked to a nucleotide sequence which, when expressed, disturbs development of the cell of the flower, seed, or embryo. A selectable marker is also included for selection of transformed cells.

Another example is the work described in U.S. Pat. No. 6,297,426, where a constitutively female sterile plant with inducible fertility was provided. The rationale behind this system was that it would allow male and female inbreds to be grown together in seed production fields, resulting in considerable cost savings in terms of land-use efficiency and in terms of field activities.

Sterility can also be caused by variations in the photoperiod which can induce female sterility, male sterility and a combination of female and male sterility by moving short day length tropical corn to long day production zones.

Punyasingh (1947) was one of the first to make an exhaustive study of triploid maize. His studied found that like in watermelon the best way of making triploids is using the tetraploid as the female. He also found that that unlike watermelon, selfed triploids have a very low level of fertility and very minor amounts of seed can be produced and that diploid pollen stimulates minor amounts of seed production (Chromosome numbers in crosses of diploid, triploid and tetraploid maize Genetics 32:541-534).

### EXAMPLES

The following examples illustrate the materials and methods used in carrying out embodiments of the invention that result in obtaining triploid maize plants. It should be understood that these Examples, while indicating preferred embodiments of the invention, are given by way of illustration only. From the above discussion and these Examples, one skilled in the art will be able to ascertain the essential characteristics of this invention, and without departing from the spirit and scope thereof, can make various changes and modifications of the invention to adapt it to various usages and conditions.

### Example 1

### Method for Producing Triploid Seed

### A) Protocol for Producing Triploid Maize in the Field.

Female Tetraploid 4N×2N=3N

Rows of Tetraploid (4N) maize inbreds, including NSL 92645 and NSL 92646 were planted on Apr. 1, 2007 in Israel, alternating with rows of any diploid maize at a planting density of 15,000 plants/acre; which is one plant every 30 cm along the row and 95 cm between adjacent rows. The plants were irrigated with drippers emitting 4 L water/hour.

The plants were fertilized with 130 Kg of NPK/acre/season.

After tassel emergence, the tassels of the diploid 2N plants were covered with paper bags and sealed.

When the shoots of the tetraploid (4N) ears were visible they were also covered with smaller paper bags and clipped shut. This process ensured that there were no foreign pollen on the diploid silks and no contamination of the 4N pollen. Since there was good nick (simultaneous blooming of female and male flowers), the ear bag of the 4N was quickly removed and the tassel bag containing pollen was quickly placed over the 4N ear and clipped closed. The resulting mature seed was triploid (3N).

### B. Laboratory Method for Producing Triploid Maize

The following protocol enables Production of Triploid Maize via Microspore Culture.

1. Mother Plant Production

Hexaploid corn plants are produced as follows:

Tetraploid maize seeds, such as NSL 92645 and NSL 92646 (available from USDA-ARS Ames, Iowa, Maize germplasm bank) are treated with colchicine (for conditions, see for example A Cytological Study of Colchicine Effects in the Induction of Polyploidy in Plants O. J. Eigsti Proceedings of the National Academy of Sciences of the United States of America, Vol. 24, No. 2 (Feb. 15, 1938), pp. 56-63) and are self-pollinated. Self pollinated seeds produced therefrom, are planted out under standard field conditions and leaf samples are taken from each plant. These samples are prepared for flow cytometry as is known in the art. Hexaploids are determined by the flow cytometry readings and those plants in the field displaying hexaploidy are used for further steps as described hereinbelow.

Hexaploid corn plants are grown in 10 liter buckets with a growth media consisting of 60% cocopeat and 40% perlite. The growth media is pretreated with Super phosphate at a rate of 12 kg/1000 liters of media. Plants are irrigated with tap water supplemented with 20-20-20. Concentrated fertilizer is prepared by mixing 25 kg of 20-20-20 with 100 liters. Two liters of the concentrate are added to each 1000 liters of irrigation water.

2. Sampling
a. The top 2-3 florets of the tassel are removed and the anthers are removed and crushed in a droplet of aceotcarmine stain.
b. The developmental stage of the microspores is then evaluated.
c. If most of the microspores are at late uninucleate to binucleate states, then the whole tassel is ready to be sampled.
d. Plants are cut at 1-2 nodes below the base of the tassel and extraneous leaves removed.
e. Shortened tassels are placed in containers with distilled water.
f. Tassels are disinfected and stored at 4° C. Tassels may be stored for 1-3 days.

3. Pretreatment
a. In a sterile laminar hood, the tassel is removed from the boot for disinfection.
b. Disinfection is done by 20% commercial bleach for 20 minutes and then rinsed 3 times with sterile water.
c. In 100 mm Petri dishes, 150-200 florets are floated in 15 ml of MMA medium (For all media compositions see Zheng et al., 2003). Zheng, M. Y., Weng, Y., Sahibzada, R., Konzak, C. F. Isolated microspore cultue in maize (Zea mays L.), production of double—haploids via induced androgenesis in Double Haploid Production in Crop Plants, M. Maluszynski, et al Kluwer Acad. Pub. 95-102, 2003.
d. Petri dishes are sealed and placed in the dark for 8-14 days at 5-10° C.

4. Isolation of Triploid (3N) Microspores
a. Induced florets are then placed in a sterile blender.
b. 50-60 mls of the isolation medium is added to the blender cup
c. Blend at 16,000 rpm for 10 seconds.
d. Slurry is filtered through a sterile 50 um mesh filter.
e. Microspores on top of the filter are rinsed 3× with 2 ml of 0.3 M mannitol and then washed off the filter into 60 mm Petri dishes with 2 ml of 0.3 M mannitol.
f. Microspores are then layered with 5 ml of 21% maltose in a sterile centrifuge cone.
g. Tube is centrifuged at 750 rpm for 2 min. Viable microspores form a band at the interphase between the mannitol and maltose.
h. Viable microspores are transferred with 3 ml of solution to another 15 ml centrifuge cone. They are centrifuged at 14,000 rpm for 1.5-2 min. The induced microspores form a band on the top of the aqueous phase.
i. Add 1 ml of the isolation medium on top of the band slowly then collect the microspores and pipette them onto another 50 um mesh filter. Allow the solution to pass.
j. Rinse microspores trapped on mesh 3× with 2 ml of induction medium. Microspores are then rinsed off the filter into 20×60 mm Petri dishes with 2 ml of induction medium IND.
k. Microspores are dispersed on the induction medium at a density of 20-70000 ml⁻¹.

5. Induction Culture.
a. 4-6 wheat ovaries are added to each Petri dish with hexaploid microspores (3N) and incubated at 27-28 C
b. The induction medium is refreshed 7 days after the culture initiation and wheat ovaries are replaced at 4 week intervals.
c. First cell divisions start 3 days after culture. Proembryoids emerge 11-14 days following culture initiation.
d. 7 days later yellowish compact embryoids/calli of 2-3 mm diameter are transferred to regeneration medium

6. Regeneration Culture
a. Emerged shoots are transferred to a regeneration medium such as Reg-III for rooting.
b. 7-10 days later regenerated 3N plantlets are ready to be hardened off.

### Example 2

### Incorporating Cytoplasmic Male Sterility (CMS) into a Tetraploid Parent

a) Back-crossing according to methods known in the art of cytoplasmic male sterility (accession number PI 600755), into a tetraploid inbred such as NSL 92645 and NSL 92646 (available from USDA-ARS Ames, Iowa, Maize germplasm bank)

b) Taking a known diploid with cytoplasmic male sterility and doubling its chromosomes by methods known in the art, such as, but not limited to, treating diploid corn plants with at least one of colchicine, heat and nitrous oxide.

### Example 3

### Preparation of Ethanol from Harvested Triploid Corn Stalks

Triploid corn plants are grown in the field from triploid corn seeds (produced in accordance with Example 1 or 2 hereinabove. Corn stalks are harvested by conventional methods known in the art. Thereafter, the stalks are crushed (or equivalent) to extract juice. The juice is typically filtered and then fermented to alcohol by methods known in the art (For example see a) Morais, P. B.; Rosa, C. A.; Linardi, V. R.; Carazza, F.; Nonato, E. A. “Production of fuel alcohol by Saccharomyces strains from tropical habitats.” Biotechnology Letters. November 1996. Vol. 18, No. 11, 1351-1356; b) Butzen et al, Crop Insights 16(7) 1-5 2006).

Typical yields of gallons ethanol/acre from these triploid stalks ranges from 1000-7000. It will be appreciated that triploid corn that is of very low female fertility behaves in a similar manner to Earless Corn Hybrids. The following examples show practical yields for Earless Corn Hybrids and thus sugar yields of triploid corn hybrids of the present invention of around 5-20% less/plant are expected. The reason for this 5-20% reduction in sugar yield is due to the very low female fertility in the triploid plants of the present invention, which may direct some of the photosynthates into a small amount of grain and hence away from the stalks.

### Example 4

### Using Inflorescence-Deficient Stalks of Earless Corn Hybrids to Produce Plants with a High Percentage of Stalk Sugars

The following data provides support for the feasibility of the approach of embodiments of the invention.

Tables 1-3 show results of field trial data performed in South Africa in 1990. Data were obtained using hybrid corn CC1 ba3-Nadel having the barren stalk3-Nadel mutation. CC1 was developed from four open-pollinated varieties: two Midwestern dents (Wilson Farm Reid Yellow Dent and Clarage) and two Southern dents (Yellow Tuxpan and Florida Laguna). Seeds of these female-sterile hybrids were planted in close proximity and were grown to produce corn plants. The corn plants were harvested and juice was extracted from the corn plants. Data presented in Tables 1-3 representing three separate field trials planted between Aug. 28, 1990 and Sep. 3, 1990. Each sample number consists of a separate analysis of five corn plants. Row 1 shows the plant density in thousands of plants per acre; row 2 shows the total wet mass of plant material obtained in kilograms; row 3 shows the total wet mass of plant stalk obtained in kilograms; row 4 shows the total mass of juice obtained in kilograms; row 5 shows the extrapolated stalk yield in tons per acre; row 6 shows the pH of the final juice; row 7 shows the Brix percentage of the juice; row 8 shows the percentage of sucrose obtained after the stalk material was pressed once; row 0 shows the percentage of sucrose obtained after the same stalk material was pressed twice; row 10 shows the percentage of sucrose in the stalk; row 11 shows the percentage of pure sucrose; row 12 shows the extrapolated yield of dry processed sucrose in tons per acre; row 13 shows the extrapolated wet leaf mass in tons per acre and row 14 shows the extrapolated stalk dry matter as a percentage of the total weight of the stalk. The data in Table 1 shows extrapolated stalk yields of 22.3 tons/acre to 27.5 tons/acre obtained with Brix percentage of juice unexpectedly ranging from 16.0% to 17.2%. Additionally, sucrose yields unexpectedly ranged from 2.4 tons/acre to 3.1 tons/acre.

TABLE 1

| | | CC1 Sample Number | | | | |
|---|---|---|---|---|---|---|
| Characteristic | | 1 | 2 | 3 | 4 | 5 |
| Plant Density | | 81 | 81 | 81 | 81 | 81 |
| Wet Mass of | Total (kg) | 3.80 | 3.98 | 4.09 | 4.34 | 4.59 |
| Sample | Stalk (kg) | 2.75 | 2.95 | 2.96 | 3.20 | 3.39 |
| | Juice (kg) | 0.82 | 0.99 | 1.00 | 1.02 | 1.02 |
| Stalk Yield (tons/acre) | | 22.3 | 23.9 | 23.9 | 25.9 | 27.5 |
| pH | | 4.9 | 5.0 | 5.1 | 5.1 | 5.1 |
| Brix % of Juice | | 16.0 | 17.2 | 16.2 | 16.8 | 17.2 |
| Percentage of | Juice | 13.1 | 13.5 | 12.6 | 13.3 | 14.1 |
| Sucrose | (1st press) | | | | | |
| | Juice | 12.3 | 12.7 | 11.9 | 12.5 | 13.3 |
| | (2nd press) | | | | | |
| | Stalk | 10.5 | 10.8 | 10.1 | 10.6 | 11.3 |

| | | | | | |
|---|---|---|---|---|---|
| Percentage of Juice Purity | 81.9 | 78.6 | 78.0 | 79.2 | 82.2 |
| Sucrose Yield (tons/acre) | 2.4 | 2.6 | 2.4 | 2.8 | 3.1 |
| Wet Leaf Mass (tons/acre) | 8.5 | 8.5 | 9.3 | 9.3 | 9.7 |
| Stalk Dry Matter | 34.6 | 33.3 | 30.9 | 31.0 | 31.0 |
| (% of stalk) | | | | | |

The data in Table 2 shows stalk yields of 23.1 tons/acre to 33.2 tons/acre obtained with Brix levels unexpectedly ranging from 17.6% to 18.2%. Additionally, sucrose yields unexpectedly ranged from 2.6 tons/acre to 3.8 tons/acre.

TABLE 2

| | | CC1 Sample Number | | | | |
|---|---|---|---|---|---|---|
| Characteristic | | 1 | 2 | 3 | 4 | 5 |
| Plant Density | | 49 | 49 | 81 | 81 | 81 |
| Wet Mass of | Total (kg) | 6.59 | 8.45 | 5.55 | 3.78 | 4.52 |
| Sample | Stalk (kg) | 5.02 | 6.46 | 4.12 | 2.87 | 3.35 |
| | Juice (kg) | 1.65 | 1.86 | 1.23 | 0.97 | 0.86 |
| Stalk Yield (tons/acre) | | 24.3 | 31.6 | 33.2 | 23.1 | 27.1 |
| pH | | 5.3 | 5.2 | 5.1 | 5.1 | 5.1 |
| Brix % of Juice | | 18.2 | 17.8 | 17.6 | 17.6 | 17.6 |
| Percentage | Juice | 14.2 | 14.2 | 14.2 | 13.9 | 14.0 |
| of Sucrose | (1st press) | | | | | |
| | Juice | 13.3 | 13.3 | 13.3 | 13.0 | 13.1 |
| | (2nd press) | | | | | |
| | Stalk | 11.4 | 11.4 | 11.4 | 11.1 | 11.2 |

| | | | | | |
|---|---|---|---|---|---|
| Percentage of Juice Purity | 78.3 | 79.8 | 80.7 | 78.9 | 79.5 |
| Sucrose Yield (tons/acre) | 2.8 | 3.6 | 3.8 | 2.6 | 3.0 |
| Wet Leaf Mass (tons/acre) | 5.5 | 9.7 | 11.7 | 7.3 | 9.3 |
| Stalk Dry Matter | 32.8 | 29.0 | 30.0 | 33.3 | 33.3 |
| (% of stalk) | | | | | |

The data in Table 3 shows stalk yields of 21.1 tons/acre to 27.1 tons/acre obtained with Brix levels unexpectedly ranging from 17.0% to 17.6%. Additionally, sucrose yields unexpectedly ranged from 2.4 tons/acre to 3.0 tons/acre.

TABLE 3

| | | CC1 Sample Number | | | | |
|---|---|---|---|---|---|---|
| Characteristic | | 1 | 2 | 3 | 4 | 5 |
| Plant Density | | 49 | 49 | 81 | 81 | 81 |
| Wet Mass of | Total (kg) | 6.89 | 6.72 | 4.88 | 4.18 | 3.80 |
| Sample | Stalk (kg) | 5.39 | 5.21 | 3.35 | 3.12 | 2.62 |
| | Juice (kg) | 1.78 | 1.52 | 1.07 | 1.12 | 0.74 |
| Stalk Yield (tons/acre) | | 26.3 | 25.5 | 27.1 | 25.1 | 21.1 |
| pH | | 5.4 | 5.3 | 5.3 | 5.3 | 5.2 |
| Brix % of Juice | | 17.0 | 17.0 | 17.6 | 17.2 | 17.4 |
| Percentage of | Juice | 13.4 | 13.5 | 14.0 | 13.7 | 13.9 |
| Sucrose | (1st press) | | | | | |
| | Juice | 12.6 | 12.7 | 13.1 | 12.9 | 13.0 |
| | (2nd press) | | | | | |
| | Stalk | 10.7 | 10.8 | 11.2 | 11.0 | 11.1 |

| | | | | | |
|---|---|---|---|---|---|
| Percentage of Juice Purity | 78.9 | 79.5 | 79.8 | 79.8 | 79.8 |
| Sucrose Yield (tons/acre) | 2.8 | 2.8 | 3.0 | 2.8 | 2.4 |
| Wet Leaf Mass (tons/acre) | 7.3 | 7.3 | 7.3 | 7.3 | 7.3 |

### Example 5

### Method of Invention Using ba3-Nadel Corn Plants—Sugar Content in Stalks of Earless Corn Hybrids is Further Increased in sugary2 Waxy Backgrounds

Table 4 shows results of field trial data performed in South Africa in 1990. Data were obtained using hybrid corn CC1, sweetXwaxy (SW/WXY), 3-WAY and waxy line cross (WXY/LC) (SW is the standard sweet corn line, sugary2 (su2); WXY is waxy endosperm; and LC is a line cross between two sister lines of the ba3-Nadel in a dent background), each having the ba3-Nadel mutation. Seeds of these female-sterile hybrids were planted in close proximity and were grown to produce corn plants. The corn plants were harvested and juice was extracted from the corn plants. The date of planting was Sep. 4, 1990. Each sample number consists of a separate plot of corn plants. Row 1 shows the plant density in hundred thousand plants per acre; row 2 shows the total wet mass of plant material obtained in kilograms; row 3 shows the total wet mass of plant stalk obtained in kilograms; row 4 shows the total mass of juice obtained in kilograms; row 5 shows the stalk yield in tons per acre; row 6 shows the pH of the final juice; row 7 shows the Brix percentage of the juice; row 8 shows the percentage of sucrose obtained after the stalk material was pressed once; row 9 shows the percentage of sucrose obtained after the same stalk material was pressed twice; row 10 shows the percentage of sucrose in the stalk; row 11 shows the percentage of juice purity; row 12 shows the yield of dry processed sucrose in tons per acre and row 13 shows the wet leaf mass in tons per acre. Stalk yields of 25.5 tons/acre to 59.6 tons/acre were obtained with Brix levels unexpectedly ranging from 16.4% to 18.8%. Additionally, sucrose yields unexpectedly ranged from 2.8 tons/acre to 5.1 tons/acre with the highest sucrose yields coming from earless plants in a sugary2 waxy background.

TABLE 4

| | | Sample | | | |
|---|---|---|---|---|---|
| Characteristic | | CC1 | SW/WXY | 3-WAY | WXY/LC |
| Plant Density | | 81 | 49 | 65 | 41 |
| Wet Mass of | Total (kg) | 3.95 | 10.9 | 6.13 | 9.22 |
| Sample | Stalk (kg) | 3.13 | 8.91 | 5.11 | 7.30 |
| | Juice (kg) | 0.80 | 2.88 | 1.75 | 2.58 |
| Stalk Yield (tons/acre) | | 25.5 | 43.3 | 33.2 | 59.6 |
| pH | | 5.3 | 5.3 | 5.2 | 5.0 |
| Brix % of Juice | | 18.0 | 18.8 | 17.8 | 16.4 |
| Percentage of | Juice | 13.6 | 14.8 | 15.0 | 12.7 |
| Sucrose | (1st press) | | | | |
| | Juice | 12.7 | 13.7 | 14.0 | 12.0 |
| | (2nd press) | | | | |
| | Stalk | 10.9 | 11.8 | 12.0 | 10.2 |

| | | | | |
|---|---|---|---|---|
| Percentage of Juice Purity | 75.4 | 78.6 | 84.3 | 77.4 |
| Sucrose Yield (tons/acre) | 2.8 | 5.1 | 4.0 | 3.0 |
| Wet Leaf Mass (tons/acre) | 6.5 | 9.7 | 6.5 | 7.7 |
| Pol. Scale (10 cm tube) | 26.1 | 28.4 | 28.9 | 24.5 |

### Example 6

### Method of Invention Using ba3-Nadel Corn Plants—Sugar Content in Stalks of Earless Corn Hybrids is Influenced by the Age of the Plant

Tables 5-7 show results of three separate field trials performed in late spring-early summer in Israel between 1987 and 1990. Planting density varied from 52.6 thousand plants/acre (Table 5) to 77.5 thousand plants/acre (Table 7). Two standard sweet corn lines, su2, containing the ba3-Nadel gene were crossed with each other. The resulting hybrids exhibited a 100% earless phenotype. Seeds of these female-sterile hybrids were planted in close proximity and were grown to produce corn plants. The corn plants were harvested and juice was extracted from the corn plants. Each sample consists of a separate analysis of five corn plants. Row 1 shows the number of days the stalks were cut after planting; row 2 shows the plant density in thousands of plants per acre; row 3 shows the total gross weight of five stalks in kilograms; row 4 shows the extrapolated gross tons of total biomass per acre; row 5 shows the net weight of the stripped stalk in kilograms; row 6 shows the extrapolated number of tons of stalk harvested per acre; row 7 shows the amount of juice as a percentage of net stalk weight; row 8 shows the pH of the juice; row 9 shows the Brix percentage of the juice; row 10 shows the percentage of purity of the juice; row 11 shows the sucrose percentage of the juice; row 12 shows the extrapolated amount of dry processed sucrose obtained in tons per acre; row 13 shows the amount of dry matter as a percentage of the total amount stalk and row 14 shows the extrapolated tons of dry matter of stalk per acre. At a density of 52.6 thousand plants per acre the percent sucrose optimized after 102 days (Table 5).

TABLE 5

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 52.6 | 52.6 | 52.6 | 52.6 | 52.6 | 52.6 |
| Total Gross Weight of | 2.3 | 1.9 | 3.1 | 2.4 | 2.0 | 2.0 |
| 5 stalks (kg) | | | | | | |
| Gross Tons per Acre | 23.9 | 19.5 | 32.8 | 25.3 | 21.1 | 20.5 |
| Net Weight (kg) | 1.7 | 1.4 | 2.4 | 1.9 | 1.6 | 1.5 |
| Tons of Stalk | 17.5 | 14.2 | 25.3 | 20.0 | 16.8 | 24.3 |
| (per acre) | | | | | | |
| Juice % | 59 | 52 | 62 | 57.5 | 41 | 60 |
| pH | 5.2 | 5.2 | 5.1 | 5.5 | 5.4 | 5.2 |
| Brix % of Juice | 11.2 | 12.9 | 17.0 | 17.0 | 18.5 | 18.5 |
| Percentage of Purity of | 65.5 | 68.1 | 67.6 | 87.2 | 84.6 | 77.8 |
| Juice | | | | | | |
| % Sucrose | 7.3 | 8.8 | 11.5 | 15.7 | 15.7 | 14.4 |
| Sucrose Yield | 0.5 | 0.8 | 1.8 | 2.4 | 2.0 | 1.6 |
| (tons/acre) | | | | | | |
| % Dry Matter | 31.3 | 28.8 | 34.8 | 39.6 | 41.1 | 43.6 |
| Tons of Dry Matter Per | 7.5 | 5.6 | 11.4 | 10.0 | 8.7 | 11.3 |
| Acre | | | | | | |

Table 6 shows that as the number of days the stalks were cut after planting increased, the Brix percentage, the percentage of sucrose obtained and the sucrose yield unexpectedly increases up to 110 days.

TABLE 6

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 61.9 | 61.9 | 61.9 | 61.9 | 61.9 | 61.9 |
| Total Gross Weight of | 2.3 | 1.9 | 2.4 | 2.6 | 2.2 | 1.5 |
| 5 stalks (kg) | | | | | | |
| Gross Tons per Acre | 28.2 | 22.9 | 29.4 | 32.2 | 26.6 | 18.5 |
| Net Weight (kg) | 1.7 | 1.3 | 1.8 | 1.9 | 1.9 | 1.3 |
| Tons of Stalk | 21.1 | 16.1 | 22.3 | 23.5 | 22.9 | 15.5 |
| (per acre) | | | | | | |
| Juice % | 69.5 | 57 | 50 | 55 | 56.5 | 45 |
| pH | 5.1 | 5.2 | 5.1 | 5.3 | 5.2 | 5.5 |
| Brix % of Juice | 11.9 | 10.7 | 16 | 17 | 20 | 17.9 |
| Percentage of Purity of | 58 | 67.1 | 70.2 | 89.3 | 88.9 | 84.5 |
| Juice | | | | | | |
| % Sucrose | 6.9 | 7.2 | 11.3 | 15.2 | 17.8 | 15.1 |
| Sucrose Yield | 0.8 | 0.8 | 1.6 | 2.7 | 3.1 | 1.8 |
| (tons/acre) | | | | | | |
| % Dry Matter | 31.1 | 29.4 | 30.7 | 39.5 | 37.8 | 39.3 |
| Tons of Dry Matter Per | 8.8 | 6.7 | 9.0 | 12.7 | 10.0 | 7.3 |
| Acre | | | | | | |

Table 7 below shows results of field trial data performed in late spring-early summer in Israel in 1990. As the number of days the stalks were cut after planting increased, the Brix percentage, the percentage of sucrose obtained and the sucrose yield unexpectedly increased, with maximum percentages and yield being reached at 102 days to 110 days after planting.

TABLE 7

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 77.5 | 77.5 | 77.5 | 77.5 | 77.5 | 77.5 |
| Total Gross Weight of | 2.3 | 2.7 | 3.2 | 2.7 | 2.0 | 1.9 |
| 5 stalks (kg) | | | | | | |
| Gross Tons per Acre | 35.5 | 41.1 | 47.6 | 41.1 | 30.2 | 28.7 |
| Net Weight (kg) | 1.7 | 2.1 | 2.5 | 2.0 | 1.7 | 1.5 |
| Tons of Stalk | 26.4 | 30.6 | 38.7 | 31.0 | 26.4 | 23.2 |
| (per acre) | | | | | | |
| Juice % | 69.5 | 62.5 | 71.2 | 69.5 | 51.0 | 54.0 |
| pH | 5.2 | 5.2 | 5.3 | 5.3 | 5.3 | 5.4 |
| Brix % of Juice | 11.7 | 13.5 | 15.0 | 17.0 | 20.0 | 18.1 |
| Percentage of Purity of | 64.4 | 77.0 | 72.4 | 88.3 | 79.1 | 73.8 |
| Juice | | | | | | |
| % Sucrose | 7.53 | 10.4 | 10.8 | 15.1 | 15.9 | 13.4 |
| Sucrose Yield | 1.2 | 2.3 | 2.8 | 3.4 | 3.0 | 2.1 |
| (tons/acre) | | | | | | |
| % Dry Matter | 31.7 | 28.1 | 31.0 | 36.6 | 39.7 | 37.8 |
| Tons of Dry Matter Per | 11.3 | 11.5 | 14.8 | 15.0 | 12.0 | 10.9 |
| Acre | | | | | | |

### Example 7

### Stalks of Eared Corn Hybrids Contain a Low Percentage of Sugar

Table 8 shows results of field data performed in late spring-early summer in Israel in 1990. Two standard sweet corn lines, su2, were crossed resulting in normal corn plant phenotypes that produced ears. Seeds of these hybrids were planted in close proximity and were grown to produce corn plants. The corn plants were harvested and juice was extracted from the corn plants. Each sample number consists of a separate plot of five corn plants. Row 1 shows the number of days the stalks were cut after planting; row 2 shows the plant density in hundred thousand plants per acre; row 3 shows the total gross weight of five stalks in kilograms; row 4 shows the extrapolated gross tons of total biomass per acre; row 5 shows the net weight stripped stalk in kilograms; row 6 shows the extrapolated tons of stalk harvested per acre; row 7 shows the amount of juice as a percentage of net stalk weight; row 8 shows the pH of the juice; row 9 shows the Brix percentage of the juice; row 10 shows the percentage of purity of the juice; row 11 shows the sucrose percentage of the juice; row 12 shows the extrapolated amount of dry processed sucrose obtained in tons per acre; row 13 shows the amount of dry matter as a percentage of the total amount of stalk and row 14 shows the tons of dry matter of stalk per acre. Brix percentages range from 5.1 to 6.8 and the sucrose yields range from 0.3 to 0.5 tons per acre. Sucrose yields peaked at 102 days after planting, but yields were one-sixth of those obtained with earless plants (see Table 7).

TABLE 8

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 77.5 | 77.5 | 77.5 | 77.5 | 77.5 | 77.5 |
| Total Gross Weight of | 1.7 | 1.9 | 2.0 | 2.1 | 1.7 | 1.6 |
| 5 stalks (kg) | | | | | | |
| Gross Tons per Acre | 26.4 | 28.7 | 31.0 | 32.6 | 26.4 | 24.0 |
| Net Weight (kg) | 1.0 | 1.1 | 1.2 | 1.2 | 1.0 | 0.8 |
| Tons of Stalk | 15.5 | 16.2 | 18.6 | 18.6 | 14.7 | 11.6 |
| (per acre) | | | | | | |
| Volume Juice % | 62.5 | 64.5 | 67.0 | 58.0 | 48.5 | 49.0 |
| pH | 5.1 | 5.2 | 5.2 | 5.3 | 5.2 | 5.4 |
| Brix % of Juice | 5.1 | 5.4 | 6.1 | 6.5 | 6.8 | 6.2 |
| Percentage of Purity of | 62.2 | 64.3 | 72.2 | 77.3 | 74.3 | 72.1 |
| Juice | | | | | | |
| % Sucrose | 3.2 | 3.5 | 3.2 | 4.4 | 5.1 | 4.5 |
| Sucrose Yield | 0.3 | 0.4 | 0.4 | 0.5 | 0.4 | 0.3 |
| (tons/acre) | | | | | | |
| % Dry Matter | 33.2 | 34.2 | 35.6 | 39.7 | 41.2 | 43.1 |
| Tons of Dry Matter Per | 8.7 | 9.8 | 11.1 | 12.9 | 10.9 | 10.4 |
| Acre | | | | | | |

### Example 8

### Stalks of Eared Corn Hybrids Contain a Lower Percentage of Sugar Than Stalks of Earless Corn Hybrids

Table 9 below shows side-by-side of data extrapolated from tables 7 and 8. Row one shows the number of days the stalks were cut after planting, row two shows the column headers for earless corn plants, designated as “EL”, and eared corn plants, designated as “E”, row three shows the gross tons of total biomass per acre, row four shows the gross tons harvested per acre, row five nine shows the tons of can harvested per acre, row six shows the Brix percentage of the juice, row seven shows the sucrose percentage of the juice and row eight shows the amount of dry processed sucrose obtained in tons per acre. Note the significant differences between the earless corn plants and the eared corn plants of each characteristic at each date. The total gross weight, the gross tons per acre, the tons of stalk harvested per acre, the Brix percentage of the juice, the percentage of sucrose of the juice and the sucrose yield of the earless plants is consistently greater than the eared plants. The Brix percentages of the earless corn plants unexpectedly range from between 2.3 to 2.9 times greater than the eared corn plants. Additionally, the sucrose yield of the earless plants unexpectedly ranges from between 4 to 7.5 times greater than the eared corn plants.

TABLE 9

| | 85 | | 89 | | 95 | | 102 | | 110 | | 115 | |
|---|---|---|---|---|---|---|---|---|---|---|---|---|
| Characteristic | EL | E | EL | E | EL | E | EL | E | EL | E | EL | E |
| Total Gross | 2.3 | 1.7 | 2.7 | 1.9 | 3.2 | 2.0 | 2.7 | 2.1 | 2.0 | 1.7 | 1.9 | 1.6 |
| Weight of 5 | | | | | | | | | | | | |
| stalks (kg) | | | | | | | | | | | | |
| Gross Tons per | 35.5 | 26.4 | 41.1 | 28.7 | 47.6 | 31.0 | 41.1 | 32.6 | 30.2 | 26.4 | 28.7 | 24.0 |
| Acre | | | | | | | | | | | | |
| Tons of Stalk | 26.4 | 15.5 | 30.6 | 16.2 | 38.7 | 18.6 | 31.0 | 18.6 | 26.4 | 14.7 | 23.2 | 11.6 |
| (per acre) | | | | | | | | | | | | |
| Brix % of Juice | 11.7 | 5.1 | 13.5 | 5.4 | 15.0 | 6.1 | 17.0 | 6.5 | 20.0 | 6.8 | 18.1 | 6.2 |
| % Sucrose | 7.53 | 3.2 | 10.4 | 3.5 | 10.8 | 3.2 | 15.1 | 4.4 | 15.9 | 5.1 | 13.4 | 4.5 |
| Sucrose Yield | 1.2 | 0.3 | 2.3 | 0.4 | 2.8 | 0.4 | 3.4 | 0.5 | 3.0 | 0.4 | 2.1 | 0.3 |
| (tons/acre) | | | | | | | | | | | | |

### Example 9

### Method of Extracting Sugar from Corn Plants Using the Juicing Method

With the present invention sugar production from maize is focused on the production of sucrose from corn stalks. The corn stalks may include some residual pieces of leaves and tassels. Corn stalks of this invention can be harvested using mechanical harvesters. After cutting, the corn stalks can be placed in a large pile and, prior to milling, cleaned. There are typically two steps to the milling process, breaking of the corn stalks and grinding of the corn stalks. Breaking of the corn stalks revolve around using knives, shredders, crushers or a combination of these processes. The grinding of the corn stalks involves using roller mills in multiple sets of three, four, five or more rollers and using conveyers to transport the crushed corn plant material from one mill to another, where it is simultaneously imbibed with water to enhance juice extraction at the next mill. The juice from the mills can then be strained to remove large particles. Next, the juice is clarified using heat (to about 200° F.) and lime (to neutralize any organic acids) and sometimes, small quantities of soluble phosphate. A heavy precipitate forms which is then separated from the juice in the clarifier. The insoluble particulate mass is called “mud” and is separated from the limed juice by gravity or centrifuge. The clarified juice contains the sugar of interest. “Cane Sugar Refining,” Cane Sugar Handbook, pp 435-499, Chen, J. P and Chow, C. C., John Wiley & Sons, Inc. (1993).

### Example 10

### Method of Extracting Sugar from Corn Plants Using the Diffusion Method

In the present invention sugar production from maize is focused on the production of sucrose from corn stalks. The corn stalks may include some residual pieces of leaves and tassels. Corn stalks of this invention are harvested using mechanical harvesters. The corn stalks are cut using knives, shredders, crushers or a combination of these processes. After cutting, conveyor belts send the corn stalks through a diffuser or extractor, where hot water is mixed in with the corn stalks to dissolve and remove the sugar. The corn stalk pulp is squeezed, where the water and sugar juice are saved and the dry pulp is removed. The water and sugar juice is then treated with milk of lime which is treated with carbon dioxide twice and then filtered to remove other non-sugars. The clarified and purified juice contains the sugar(s) of interest. “Cane Sugar Refining,” Cane Sugar Handbook, pp 435-499, Chen, J. P and Chow, C. C., John Wiley & Sons, Inc. (1993)

### Example 11

### Industrial Methods of Extracting Ethanol from Corn Plants

There are several steps involved in the process of producing ethanol from the juice extracted from the corn stalks, where the corn stalks may include residual pieces of leaves and tassels. Ethanol was produced through a biochemical processes based on fermentation using the juice or molasses as a feedstock (or a mixture of the juice and molasses). The sugars were transformed into alcohol using yeasts as the catalyst. Fermentation can take anywhere from four to twelve hours and liberate a significant amount of carbon dioxide and heat. The fermentation process can be conducted in batch or continuously, using open or closed fermentation tanks to produce ethanol. The resulting ethanol can then be distilled from other by-products in order to achieve any level of purity. Stockholm Environment Institute, Sugarcane Resources for Sustainable Development: A Case Study in Luena, Zambia, April 2001.

Additional methods of producing ethanol from sugar containing substrates are given in U.S. Pat. Nos. 4,326,036, 4,560,659, 4,738,930, 4,876,196 and U.S. Pub. No. 20030143704, all of which are herein incorporated by reference.

It should be understood that triploid corn that is of very low female fertility behaves in a similar manner to Earless Corn Hybrids. The following examples show practical yields for Earless Corn Hybrids and thus sugar yields of triploid corn hybrids of the present invention of around 5-20% less/plant are expected. The reason for this 5-20% reduction in sugar yield is due to the very low female fertility in the triploid plants of the present invention, which may direct some of the photosynthates into a small amount of grain and hence away from the stalks.

### Example 12

### Comparison of Brix Percentage Levels Between Earless and Eared Hybrid Corn 111 within One Day of Anthesis

Table 11 shows the Brix percentage levels for various nodes for both earless and eared hybrid 111 corn plants taken in Kfar Pines, Israel within one day of anthesis. Hybrid 111 corn is a yellow dent corn. The hybrid corn 111 plants were planted on Mar. 28, 2007 and the Brix readings were taken on Jun. 14, 2007. Column 1 shows the various nodes, column 2 shows the Brix percentage levels for earless inbred corn 111 plants that were grown in a greenhouse, column 3 shows the Brix percentage levels of earless inbred corn 111 plants grown in the field and column 4 shows the Brix percentage levels of eared inbred corn 111 plants. Note that nodes 1-2 are the nodes closest to ground level, while nodes 11-12 are the highest (farthest from ground level). The data show that there are minor differences in accumulated sugars between the earless and eared corn plants within one day of anthesis, with the differences between the field eared and field earless plants being 1.38% which is equivalent to a 9% total sugars difference. This difference will substantially increase as more sugar resources in the stalk of the normal-type corn plants with ears are used in the reproductive cycle.

TABLE 11

| | Brix Percentage of Each Corn Type | | |
|---|---|---|---|
| | 111 earless - | 111 earless - | 111 normal with |
| Nodes | greenhouse | field | ears - field |
| 1-2 | 13.50 | 14.80 | 11.96 |
| 3-4 | 9.50 | 16.56 | 15.68 |
| 5-6 | 13.50 | 16.62 | 16.30 |
| 7-8 | 18.25 | 17.70 | 15.82 |
| 9-10 | 18.25 | 17.44 | 16.86 |
| 11-12 | 12.00 | 16.64 | 14.90 |
| All Nodes | 14.17 | 16.63 | 15.25 |

Table 12 shows the Brix percentage levels for various nodes for both earless and eared hybrid 302 corn plants taken in Kfar Pines, Israel 8 days past anthesis. Hybrid 302 corn is a yellow flint dwarf corn plant. The hybrid corn 302 plants were planted on Mar. 28, 2007 and the Brix readings were taken on Jun. 18, 2007. Column 1 shows the various nodes, column 2 shows the Brix percentage levels for earless hybrid corn 302 plants grown in the field and column 3 shows the Brix percentage levels of eared hybrid corn 302 plants grown in the field. Note that nodes 1-2 are the nodes closest to ground level, while nodes 11-12 are the highest (farthest from ground level). Data show a difference of 2.9% in Brix percentage levels between the eared and earless hybrid 302 corn plants, where the difference will continue to be observed until senescence. When the data from Tables 11 and 12 are taken together, the difference in Brix percentage levels increases because as the grain matures and stalk sugar resources are used for all the component parts of the female reproductive system which includes but is not limited to, the shank, cob, silk, embryos-seed, and husks.

TABLE 12

Brix Percentage of Each Corn Type

| | 302 yellow flint dwarf | 302 yellow flint dwarf |
|---|---|---|
| Nodes | earless - field | with ears - field |
| 1-2 | 10.52 | 8.40 |
| 3-4 | 14.28 | 11.20 |
| 5-6 | 15.08 | 11.76 |
| 7-8 | 14.80 | 11.74 |
| 9-10 | 15.06 | 12.04 |
| 11-12 | 15.18 | 12.36 |
| All Nodes | 14.15 | 11.25 |

### Net Energy Balance

A comparative study was performed relating to the net energy ratio using corn grain derived ethanol, CO₂ intensive, cellulosic and Corn Cane. Corn Cane calculations are based on 11,000 liters ethanol/acre. The results of the study appear in Table 13 hereinbelow.

TABLE 13
Net energy ratios results from different energy sources

| | | | | | | | Corn | | |
|---|---|---|---|---|---|---|---|---|---|
| | | | | | | | Stalks | | |
| | | | | | | | Nadel | | |
| Net Energy | | | | | | Current | et al. | CO₂ | |
| Summary* 0 | | Shapouri*² | | de | Wang*⁵ | Data*⁶ | US Pat | Intensive*⁸ | Cellulosic*⁹ |
| adjusted for | Pimentel*¹ | Corn | Graboski*³ | Oliviera*³ | Corn | Corn | Appl. | Corn | Switch- |
| comparability | Corn grain | grain | Corn grain | Corn grain | grain | grain | 60/815,953 | grain | grass |
| Nitrogen | 54 | 57 | 51 | 53 | 59 | 57 | 114 | 57 | 49 |
| (MJ/kg) | | | | | | | | | |
| N | 153 | 150 | 151 | 146 | 147 | 150 | 300 | 148 | 50 |
| Application | | | | | | | | | |
| rate (kg/ha) | | | | | | | | | |
| Phosphorus | 17 | 9 | 2 | 7 | 20 | 9 | 6 | 9 | 11 |
| (MJ/kg) | | | | | | | | | |
| P2O5 | 65 | 64 | 55 | 64 | 51 | 64 | 128 | 39 | 2 |
| application | | | | | | | | | |
| (kg/ha) | | | | | | | | | |
| Potassium | 14 | 7 | 5 | 6 | 9 | 7 | 14 | 7 | 5 |
| (MJ/kg) | | | | | | | | | |
| K2O | 77 | 99 | 64 | 88 | 61 | 99 | 198 | 24 | 3 |
| application | | | | | | | | | |
| (kg/ha) | | | | | | | | | |
| Lime | 1 | 0 | 0.1 | 2 | 0 | 0 | 0 | 0 | 0 |
| (MJ/kg) | | | | | | | | | |
| Lime | 1,121 | 18 | 2,959 | 275 | 0 | 448 | 0 | 448 | 0 |
| application | | | | | | | | | |
| (kg/ha) | | | | | | | | | |
| Herbicide | 418 | 356 | 303 | 252 | 323 | 356 | 356 | 356 | 322 |
| (MJ/kg) | | | | | | | | | |
| Herbicide | 6.2 | 2.8 | 2.4 | 3.0 | 2.8 | 2.8 | 5.0 | 2.2 | 0.4 |
| application | | | | | | | | | |
| rate (kg/ha) | | | | | | | | | |
| Insecticide | 418 | 358 | 303 | 269 | 338 | 358 | 500 | 358 | 377 |
| (MJ/kg) | | | | | | | | | |
| Insecticide | 2.80 | 0.21 | 0.15 | 1.00 | 0.21 | 0.21 | 1.00 | 0.18 | 0 |
| (kg/ha) | | | | | | | | | |
| Seed (MJ/kg) | 104 | 10 | 10 | 98 | 0 | 10 | 20 | 10 | 0 |
| Seed rate | 21 | 24 | 21 | 21 | 0 | 24 | 48 | 22 | 0 |
| (kg/ha) | | | | | | | | | |
| Transportation | 707 | 73 | 738 | 0 | 168 | 504 | 3,000 | 437 | 36 |
| of inputs, | | | | | | | | | |
| summary | | | | | | | | | |
| (MJ/ha) | | | | | | | | | |
| Transport | 0.49 | 0.21 | 0.23 | 0 | 0.64 | 0.64 | 1.00 | 0.64 | 0.64 |
| energy | | | | | | | | | |
| (MJ/kg) | | | | | | | | | |
| Gasoline | 1,695 | 1,277 | 1,290 | 960 | 1,501 | 1,277 | 1,227 | 789 | 0 |
| (MJ/ha) | | | | | | | | | |
| Diesel | 4,197 | 2,719 | 3,205 | 2,907 | 4,310 | 2,719 | 16,314 | 4,922 | 3,737 |
| (MJ/ha) | | | | | | | | | |
| Natural gas | 0 | 670 | 597 | 504 | 1,626 | 670 | 0 | 2,627 | 0 |
| (MJ/ha) | | | | | | | | | |
| LPG (MJ/ha) | 0 | 765 | 1,357 | 1,512 | 1,072 | 765 | 1,500 | 918 | 0 |
| Electricity | 143 | 820 | 1,571 | 657 | 225 | 820 | 0 | 3,721 | 618 |
| (MJ/ha) | | | | | | | | | |
| Energy used | 1,339 | 49 | 0 | 0 | 0 | 49 | 90 | 329 | 0 |
| in irrigation | | | | | | | | | |
| (MJ/ha) | | | | | | | | | |
| Farm labor | 0 | 574 | 628 | 0 | 0 | 574 | 1,108 | 0 | 0 |
| (MJ/ha) | | | | | | | | | |
| Farm | 4,259 | 320 | 320 | 320 | 320 | 320 | 1,500 | 320 | 320 |
| machinery | | | | | | | | | |
| (MJ/ha) | | | | | | | | | |
| Inputs | 74 | 74 | 74 | 74 | 74 | 74 | 74 | 74 | 74 |
| packaging | | | | | | | | | |
| (MJ/ha) | | | | | | | | | |
| Total | 30,179 | 18,434 | 19,220 | 19,131 | 20,571 | 18,921 | 26,504 | 24,210 | 7,411 |
| Agricultural | | | | | | | | | |
| Phase | | | | | | | | | |
| (MJ/ha) | | | | | | | | | |
| Biorefinery | 0 | | 0 | 0 | | | | | |
| Phase | | | | | | | | | |
| Transportation | 1.35 | 0.59 | 0.49 | 0.60 | 0.64 | 0.59 | 0.60 | 2.80 | 0.63 |
| of | | | | | | | | | |
| feedstock to | | | | | | | | | |
| biorefinery | | | | | | | | | |
| (MJ/L) | | | | | | | | | |
| Primary | 11 | 14 | 15 | 13 | 0 | 0 | 0 | 0 | 0 |
| energy | | | | | | | | | |
| (MJ/L) | | | | | | | | | |
| Coal (MJ/L) | 0 | 0 | 0 | 0 | 7 | 8.3 | 0 | 13 | 0 |
| Natural gas | 0 | 0 | 0 | 0 | 4.6 | 5.5 | 0 | 0 | 0 |
| (MJ/L) | | | | | | | | | |
| Diesel | 0 | 0 | 0 | 0 | 0 | 0 | 0 | 0 | 0.06 |
| (MJ/L) | | | | | | | | | |
| Biomass | 0 | 0 | 0 | 0 | 0 | 0 | 0 | 0 | 26 |
| (MJ/L) | | | | | | | | | |
| Capital | 0.13 | 0.13 | 0.13 | 0.13 | 0.13 | 0.13 | 0.13 | 0.13 | 0.44 |
| (plant and | | | | | | | | | |
| equipment) | | | | | | | | | |
| (MJ/L) | | | | | | | | | |
| Process | 0.38 | 0.38 | 0.38 | 0.38 | 0.38 | 0.38 | 0.38 | 0.38 | 0.29 |
| water (MJ/L) | | | | | | | | | |
| Effluent | 0.29 | 0.29 | 0.29 | 0.29 | 0.29 | 0.29 | 0.29 | 0.29 | 0.29 |
| restoration | | | | | | | | | |
| (BOD energy | | | | | | | | | |
| cost in MJ/L) | | | | | | | | | |
| Totals | | | | | | | | | |
| Crop yield | 8,655 | 8,746 | 8,799 | 7,850 | 7,846 | 8,746 | 100,000 | 8,389 | 13,450 |
| (kg/ha) | | | | | | | | | |
| Biorefinery | 0.37 | 0.40 | 0.39 | 0.39 | 0.39 | 0.40 | 0.25 | 0.40 | 0.38 |
| yield (L/kg | | | | | | | | | |
| corn) | | | | | | | | | |
| Ethanol yield | 3,217 | 3,463 | 3,474 | 3,038 | 3,060 | 3,463 | 10143 | 3,322 | 5,135 |
| per land area | | | | | | | | | |
| (L/ha) | | | | | | | | | |
| Fuel energy | 69,060 | 73,424 | 73,553 | 64,101 | 64,870 | 73,424 | 220,229 | 70,431 | 108,855 |
| yield per | | | | | | | | | |
| land area | | | | | | | | | |
| (MJ/ha) | | | | | | | | | |
| Agricultural | 9.4 | 5.3 | 5.5 | 6.3 | 6.7 | 5.5 | 1.0 | 7.3 | 1.4 |
| energy | | | | | | | | | |
| (MJ/L) | | | | | | | | | |
| Biorefinery | 17.0 | 15.2 | 16.6 | 14.1 | 12.6 | 15.2 | 1.4 | 16.7 | 28.0 |
| energy | | | | | | | | | |
| (MJ/L) | | | | | | | | | |
| Corn grain | 0 | 0 | 0 | 0 | 0 | 0 | 25 | 0 | 26 |
| Input energy | 26.4 | 20.6 | 22.2 | 20.4 | 19.3 | 20.7 | 2.4 | 24.0 | 3.2 |
| (MJ/L) | | | | | | | | | |
| Reported HV | 21 | 21 | 21 | 21 | 21.2 | 21 | 21 | 21 | 21 |
| of ethanol | | | | | | | | | |
| (MJ/L) | | | | | | | | | |
| Coproduct | 1.9 | 7.3 | 4.1 | 4.1 | 4.0 | 4.1 | 7.5 | 4.1 | 4.8 |
| credits | | | | | | | | | |
| (MJ/L) | | | | | | | | | |
| Coproducts | 7% | 36% | 19% | 20% | 21% | 20% | 307% | 17% | 16% |
| as % of total | | | | | | | | | |
| energy | | | | | | | | | |
| Output | 23.3 | 28.5 | 25.3 | 25.2 | 25.2 | 25.3 | 28.7 | 25.3 | 26.0 |
| Energy | | | | | | | | | |
| (MJ/L) | | | | | | | | | |
| Net energy | 03.1 | 7.9 | 3.1 | 4.8 | 5.9 | 4.6 | 26.2 | 1.3 | 22.8 |
| value, NEV | | | | | | | | | |
| (MJ/L) | | | | | | | | | |
| Net energy | 0.9 | 1.4 | 1.1 | 1.2 | 1.3 | 1.2 | 11.5 | 1.1 | 8.2 |
| ratio | | | | | | | | | |

*¹Ethanol Production Using Corn, Switchgrass, and Wood; Bio-diesel Production Using Soybean and Sunflower, Natural Resources Review
*²The 2001 Net Energy Balance of Corn Ethanol, Presented at the Corn Utilization and Technology Conference, June 709, 2004, Indianapolis, IN
*³Fossil Energy Use in the Manufacture of Corn Ethanol, Prepared for the National Corn Growers Association
*⁴Ethanol as Fuel: Energy, Carbon Dioxide Balances, and Ecological Footprint, BioScience July 2005

Table 14 shows further results from the study of Table 13 and relates to the net energy ratio, renewability of the energy source and economic feasibility.

TABLE 14
Net Energy Ratio vs. Renewability

| | Source | | | | | |
|---|---|---|---|---|---|---|
| | Pimentel | Shapouri | IEA | Nadel | Cellulosic | IEA |
| | 2001*⁷ | 2002*⁸ | 2007* | 2007 | 2007*⁹ | 2006*¹⁰ |
| | Crop | | | | | |
| | Corn | Corn | | Corn | Switch | Sugar |
| | grain | grain | Cassava | Cane | grass | cane |
| NEB | 0.9 | 1.4 | 10.2 | 11.5 | 20 | 8.3 |
| Days to | 130 | 130 | 240 | 95 | 183 | 365 |
| Mature | | | | | | |
| Seasons/ | 1 | 1 | 1 | 1-3 | 1 | 0.66 |
| Year | | | | | | |
| Econom- | (−) | (+) | (+) | (+) | (−) | (+) |
| ical | | | | | | |

*⁷www.news.cornell.edu/Chronicle/01/8.23.01/Pimentel-ethanol.html - 7k
*⁸www.heartland.org/Article.cfm?artId=16324
*⁹http://biopact.com/2007/10/caltech-ventures-to-produce-ethanol.html
*¹⁰http://www.biopact.com/2006/10/brazilian-ethanol-is-sustainable-and.html

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

In the claims, the word “comprise”, and variations thereof such as “comprises”, “comprising” and the like indicate that the components listed are included, but not generally to the exclusion of other components.

What is claimed is:

1. A method for producing a high carbohydrate triploid corn plant, the method comprising:
a. combining two parent inbreds of different ploidy levels, wherein one parent inbred is a tetraploid (4N) and the other parent is a diploid (2N) so as to produce a triploid hybrid corn seed; and
b. cultivating said triploid hybrid corn seed to form a triploid corn plant.

2. The method of claim 1, wherein the combining step comprises crossing a tetraploid female with a male diploid to form the triploid hybrid corn seed.

3. The method of claim 1, wherein the combining step comprises crossing a tetraploid male with a diploid female to form the triploid hybrid corn seed.

4. The method of claim 1, wherein said triploid corn plant has a female fertility level of less than 20%.

5. The method of claim 1, wherein said triploid corn plant has a female fertility level of less than 10%.

6. The method of claim 4, wherein said female fertility level enhances carbohydrate storage in stalks of said triploid corn plant.

7. The method of claim 1, wherein said triploid corn plant has at least one of the following properties:
a. less than 20% of seed count of a normal diploid corn plant; and
b. a calorific value of around 10-15% less than an earless diploid corn plant.

8. The method of claim 1, wherein a stalk of the triploid corn plant comprises between 10% and 90% more carbohydrates than a stalk of a normal diploid corn plant.

9. The method of claim 8 wherein the carbohydrate comprises at least one sugar.

10. The method of claim 9, wherein said at least one sugar is selected from the group comprising monosaccharides and disaccharides.

11. The method of claim 10, wherein said disaccharide comprises sucrose.

12. The method of claim 11, wherein said triploid corn plant comprises at least 3% sucrose.

13. The method of claim 12, wherein a liquid extract of stalks from said triploid corn plant comprises at least 15° Brix.

14. The method of claim 1, wherein a sucrose yield per acre of a crop of said triploid corn plant is at least 25% more than a sucrose yield per acre for a crop of normal diploid corn plant.

15. The method of claim 14, wherein the sucrose yield per acre of said triploid corn plant is at least 25% more than a sucrose yield of a normal diploid corn plant.

16. The method of claim 1 further comprising producing fodder from said triploid plants.

17. The method of claim 16, wherein the fodder has an increased level of digestibility, relative to fodder produced from normal diploid corn plants.

18. The method of claim 1, wherein said cultivating step comprises planting a plurality of said triploid hybrid corn seeds at an average spacing of less than 0.4 meter.

19. A method according to claim 1, wherein said triploid corn plant matures within 100 days.

20. The method of claim 1, further comprising the step of extracting juice from stalks of said triploid plant.

21. The method of claim 20, further comprising fermenting the juice.

22. The method of claim 20, further comprising the step of producing molasses from said juice.

23. The method of claim 20, further comprising distilling said at least one fermentation product to produce spirits.

24. The method of claim 20, further comprising treating said juice to form crystallized sugar.

25. A method according to claim 20 further comprising producing corn bagasse from said stalks.

26. The method for producing alcohols comprising the steps of:
a. producing the triploid corn plant according to the method of claim 1;
b. extracting juice from stalks of said triploid corn plant; and
c. fermenting said juice to produce at least one alcohol.

27. The method according to claim 26, wherein said at least one alcohol comprises ethanol.
